(19) United States
(12) Patent Application Publication
(10) Pub. No.: US 2007/0197452 A1
(43) Pub. Date: Aug. 23, 2007

(54) Treatment of amyloid-related diseases

(76) Inventor: JoAnne McLaurin, East York (CA)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/706,989

(22) Filed: Feb. 16, 2007

Related U.S. Application Data
(60) Provisional application No. 60/774,918, filed on Feb. 17, 2006.
(60) Provisional application No. 60/811,587, filed on Jun. 7, 2006.

(51) Int. Cl. A61K 31/22; A61K 31/13; A61K 31/045

(52) U.S. Cl. 514/23; 514/548; 514/729; 514/659

(57) ABSTRACT

The invention provides compositions, methods and uses comprising a scyllo-inositol compound of the formula Ia or Ib

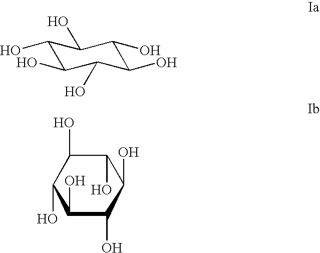

or a compound of the formula Ia or Ib wherein one, two or three hydroxyl groups are replaced by substituents with retention of configuration, or pharmaceutically acceptable salts thereof, in a therapeutically effective amount to provide beneficial effects in the treatment of an amyloid-related disease.

This application claims the benefit of priority under 35 U.S.C. § 119(e) to U.S. Provisional Application 60/744,918, filed Feb. 17, 2006 and U.S. Provisional Application 60/811,587, filed Jun. 7, 2006, incorporated herein by reference in full.

### FIELD OF THE INVENTION

The invention relates generally to scyllo-inositol compounds and compositions, and methods and uses of the compositions, in particular methods for treating amyloid-related diseases.

### BACKGROUND OF THE INVENTION

Multiple lines of evidence suggest that the accumulation of neurotoxic oligomeric/protofibrillar aggregates of amyloid β-peptide (Aβ) is a central event in the pathogenesis of Alzheimer disease (AD) [1, 2]. This has led to attempts to develop therapies based upon blocking the generation of Aβ (e.g., with β- or γ-secretase inhibitors), accelerating its removal, or preventing its aggregation and toxicity. The potential utility of anti-Aβ therapies for AD has received tentative support from a clinical trial of a vaccine, which suggested clinical and neuropathological improvement in a small cohort of AD patients [24, 25]. However, the anti-Aβ vaccine also induced a T-cell-mediated meningo-encephalitis in some patients which renders this particular vaccine unsuitable for widespread clinical use [36]. Nevertheless, Aβ vaccines have been shown in some mouse models to act via antibody-mediated inhibition of Aβ fibrillogenesis and toxicity [21, 37, 38]. Thus, it would be desirable to identify small molecule inhibitors of Aβ-aggregation that would avoid the potential risks of immunotherapy.

### SUMMARY OF THE INVENTION

The invention provides a composition, in particular a pharmaceutical composition, comprising a scyllo-inositol compound that provides beneficial effects in the treatment of an amyloid-related disease. In an aspect the invention provides a pharmaceutical composition, comprising one or more scyllo-inositol compound that provides beneficial effects, in particular sustained beneficial effects, following treatment. The beneficial effects provided by a composition of the invention can include enhanced therapeutic effects, in particular sustained therapeutic effects.

The invention also provides a pharmaceutical composition intended for administration to a subject to provide beneficial effects, in particular sustained beneficial effects, comprising a scyllo-inositol compound, in particular a pure scyllo-inositol compound, more particularly a substantially pure scyllo-inositol compound, optionally together with one or more pharmaceutically acceptable carriers, excipients, or vehicles.

The invention also provides a pharmaceutical composition for the treatment of a disorder and/or disease comprising a therapeutically effective amount of a scyllo-inositol compound to provide a sustained beneficial effect in a pharmaceutically acceptable carrier, excipient, or vehicle.

In an aspect, a pharmaceutical composition comprising a scyllo-inositol compound is provided which has been adapted for administration to a subject to provide sustained beneficial effects to treat an amyloid-related disease. In an embodiment, the composition is in a form such that administration to a subject suffering from an amyloid-related disease results in improved cognitive function, reduced vascular load, reduced astrogliosis, reduced amyloid burden, reduced microgliosis, and/or improved survival. In particular, the composition is in a form that results in improved cognitive function, reduced vascular load, reduced astrogliosis, reduced amyloid burden, reduced microgliosis, and/or improved survival in the subject, in particular for a sustained period of time after cessation of treatment.

The present invention is directed to compositions comprising a scyllo-inositol compound that provides beneficial effects, in particular sustained beneficial effects, in the treatment of an amyloid-related disease, more particularly Alzheimer's disease.

In another aspect, the invention features a composition comprising a scyllo-inositol compound in a dosage effective for improving cognitive function, reducing vascular load, reducing astrogliosis, reducing amyloid burden, reducing microgliosis, and/or improving survival in the subject, in particular for a sustained period following administration of the compound. The composition can be in a pharmaceutically acceptable carrier, excipeint, or vehicle.

The invention additionally provides a method of preparing a stable pharmaceutical composition comprising one or more scyllo-inositol compound adapted to provide beneficial effects, preferably sustained beneficial effects, following treatment. The invention further provides a method of preparing a stable pharmaceutical composition comprising a therapeutically effective amount of one or more pure, in particular substantially pure, scyllo-inositol compound adapted to provide beneficial effects, preferably sustained beneficial effects, following treatment. After compositions have been prepared, they can be placed in an appropriate container and labelled for treatment of an indicated condition. For administration of a composition of the invention, such labelling would include amount, frequency, and method of administration.

A scyllo-inositol compound for use in the present invention may be in the form of a prodrug that is converted in vivo to an active compound. By way of example, a scyllo-inositol compound may comprise a cleavable group that is cleaved after administration to a subject to provide an active (e.g. therapeutically active) compound, or an intermediate compound that subsequently yields the active compound. The cleavable group may be an ester that can be removed either enzymatically or non-enzymatically.

A scyllo-inositol compound for use in the present invention may optionally comprise a carrier interacting with the compound. A carrier may include a polymer, carbohydrate, or peptide, or combinations thereof. A carrier may be substituted, for example, with one or more alkyl, halo, thiol, hydroxyl, or amino group.

In an aspect, the invention provides a dietary supplement composition comprising one or more scyllo-inositol compound or nutraceutically acceptable derivatives thereof. In an aspect, the invention provides a dietary supplement for mammalian consumption, particularly human consumption for the purpose of improving memory comprising a scyllo-inositol compound or nutraceutically acceptable derivatives thereof. In another aspect, the invention provides a supplement comprising a scyllo-inositol compound or nutraceutically acceptable derivatives thereof for slowing the deterioration of mental processes and improving memory, in particular short-term memory, of individuals who have taken the supplement. A dietary supplement of the invention is preferably pleasant tasting, effectively absorbed into the body and provides substantial therapeutic effects.

The invention also provides methods to make commercially available formulations which contain a scyllo-inositol compound.

In an aspect, scyllo-inositol compounds, in particular pure or substantially pure scyllo-inositol compounds, and compositions of the invention may be administered therapeutically or prophylactically to treat an amyloid-related disease.

The invention also contemplates the use of a composition comprising at least one scyllo-inositol compound for the preparation of a medicament for preventing and/or treating any amyloid-related disease. The invention additionally provides uses of a pharmaceutical composition of the invention in the preparation of medicaments for the prevention and/or treatment of an amyloid-related disease.

The invention provides a method for treating and/or preventing amyloid-related diseases in a subject comprising administering to the subject a therapeutically effective amount of one or more scyllo-inositol compound to provide beneficial effects. In an aspect the invention provides a treatment which results in sustained beneficial effects following treatment.

This invention also includes a regimen for supplementing a healthy human's diet by administering a scyllo-inositol compound or a dietary supplement comprising a scyllo-inositol compound or a nutraceutically acceptable derivative thereof, and an acceptable carrier, to the human. The invention further includes a regimen for supplementing a healthy human's diet by administering daily to the human a scyllo-inositol compound or a nutraceutically acceptable derivative thereof.

The invention also provides a kit comprising one or more scyllo-inositol compound or a pharmaceutical composition of the invention. In an aspect, the invention provides a kit for preventing and/or treating a disorder and/or disease, containing a composition comprising one or more scyllo-inositol compound, a container, and instructions for use. The composition of the kit can further comprise a pharmaceutically acceptable carrier, excipient, or vehicle. In an aspect, a pharmaceutical kit is provided comprising one or more containers filled with a pharmaceutical composition of the invention, and a notice in the form prescribed by a governmental agency regulating the labeling, manufacture, use or sale of the pharmaceutical composition, which notice reflects approval by the agency of manufacture, use, or sale for human administration, in particular for human administration in the treatment of Alzheimer's disease, dementia, or mild cognitive impairment.

These and other aspects, features, and advantages of the present invention should be apparent to those skilled in the art from the following drawing and detailed description.

### DESCRIPTION OF THE DRAWINGS

The invention will be better understood with reference to the drawings in which:

FIG. 1. Spatial reference memory test in six month old mice following 28 days of treatment, beginning at five months of age (n=10 mice per treatment arm) was performed. The performance of epi-cyclohexanehexol treated TgCRND8 mice was not different from untreated TgCRND8 littermates (p=0.27; FIG. 1A) and remained impaired with respect to non-Tg littermates (F1,14=11.7, p=0.004; FIG. 1C). In contrast, scyllo-cyclohexanehexol treated TgCRND8 mice were significantly better than untreated TgCRND8 littermates (p=0.01; FIG. 1B) and were indistinguishable from non-Tg littermates (F1,13=2.9, p=0.11; FIG. 1D). The probe trial, using annulus crossing index, demonstrated that scyllo-cyclohexanehexol treated mice were not statistically different from non-Tg littermates (p=0.64; FIG. 1E). Vertical bars represent s.e.m. After one month of scyllo-cyclohexanehexol treatment, mice had a lower plaque burden compared to control animals with a high plaque burden in the hippocampus (FIG. 1F, FIG. 1G). Plaque burden was identified using anti-Aβ antibody (brown) and astrocytes are labeled using anti-GFAP antibody (red). Scale bar 300 μm.

FIG. 2. Dot blot analyses of soluble oligomeric Aβ in scyllo-cyclohexanehexol and epi-cyclohexanehexol treated and untreated TgCRND8 mice (FIG. 2A). Soluble proteins isolated from 4 representative four and six month old untreated and treated TgCRND8 mice from the prophylactic study, and from the five month old treatment groups, untreated and treated were applied to nitrocellulose and probed with oligomer-specific antibody followed by re-probing with 6E10. Synthetic Aβ42, monomeric (bottom row: lane 1 and 2) and fibrillar (lane 3 and 4) were used as negative controls for the oligomer-specific antibody, which only recognizes soluble aggregates. 6E10 recognises all Aβ species (bottom lane, right four lanes). Long-term potentiation is blocked by soluble Aβ oligomers (FIG. 2B; green squares) and rescued by scyllo-cyclohexanehexol treatment (FIG. 2B; blue circles). LTP is unaffected by scyllo-cyclohexanehexol treated 7PA2 culture medium which contains Aβ oligomers (FIG. 2C; red squares; same data as in FIG. 2B) and plain CHO medium which lacks oligomers (FIG. 2C; blue circles).

FIG. 3. Cyclohexanehexols improve behaviour in TgCRND8 mice. Spatial reference memory version of the Morris Water Maze test in TgCRND8 mice (n=8-10 per treatment arm) was used as a measure of cognition. At four months of age, non-treated TgCRND8 mice show cognitive impairment relative to AZD-102 (A) and AZD-103(B) treated mice (F2,26=3.99, p=0.03). AZD-102 treated mice (C) were significantly different from treated and untreated non-Tg mice (F1,18=11.7, p=0.004), whereas AZD-103 treated mice (D) approached that of non-Tg mice (F1,17=2.89, p=0.97). At six months of age, non-treated TgCRND8 show cognitive impairment relative to non-Tg controls (F1,30=31.16, p<0.001) and AZD-102 (E) and AZD-103 (F) treated mice (F2,36=4.1, p<0.02). The performance of both AZD-102 treated TgCRND8 mice (F1,21=2.35, p=0.14; G) and AZD-102 approached that of non-Tg littermates (F1,22=3.26, p=0.44; D). Non-Tg littermate behaviour was not affected by either AZD-102 (G) or AZD-103 (H) treatment (F2,37=0.83, p=0.45).

FIG. 4. Cyclohexanehexols improve pathological characteristics in TgCRND8 mice. Vascular Aβ burden was quantitated on serial sagittal sections in treated and untreated TgCRND8 mice. TgCRND8 mice have a significant vascular Ab burden that is associated with small and medium sized vessels; the load is decreased in AZD-103 treated TgCRND8 mice (A). AZD-103 treatment significantly decreased the total vascular load in comparison to untreated and epi-inositol treated TgCRND8 mice. Tg CRND8 mice demonstrate an astrogliotic response to increased Aβ levels within the CNS and treatment with AZD-102 decreased the percent brain area covered in astrogliosis at both 4- and 6-months of age (B). AZD-103 treatment decreased the astrogliotic response to a greater extent at both ages (B). Similarly, microgliosis has been correlated with plaque burden in the TgCRND8 mice (C). Treatment with both AZD-102 and to a greater extent with AZD-103 decreased the percent brain area covered with microgliosis. Kaplan Meier cumulative survival plot demonstrates the increased survival of TgCRND8 mice after treatment with AZD-103 (light gray circles; p=0.02) in comparison to untreated TgCRND8 mice (gray circles). AZD-102 did not significantly improve survival (black circles) (D). ANOVA *p<0.05, **p<0.001.

FIG. 5. At six months of age, the plaque burden and astrogliosis in TgCRND8 mice untreated, AZD-102 and AZD-103 treated mice were examined. Control animals have a high plaque load and astrogliosis in the hippocampus and cerebral cortex. Higher magnification demonstrates that astrocytic activation is not only associated with plaque load. AZD-102 treatment has a modest effect on amyloid burden with a decrease in astrogliosis. AZD-103 treatment significantly decreased amyloid burden and gliosis. Astrocytes labeled with anti-GFAP antibody (red) and plaque burden identified with anti-AP antibody (brown). Scale Bar 300 μm or 62.5 μm.

FIG. 6. Spatial reference memory test in six month old mice following 28 days of treatment, beginning at five months of age (n=10 mice per treatment arm) was performed. The performance of AZD-102 treated TgCRND8 mice was not different from untreated TgCRND8 littermates (p=0.27;A) and remained impaired with respect to non-Tg littermates (F1,14=11.7, p=0.004; C). In contrast, AZD-103 treated TgCRND8 mice were significantly better than untreated TgCRND8 littermates (p=0.01; B) and were indistinguishable from non-Tg littermates (F1,13=2.9, p=0.11; D).

FIG. 7. A, B, C and D. A cue test was performed at the end of the spatial memory version of the Morris Water Test. A flag was placed on the platform and the path length required to reach the platform is comparable for all treatment groups (p=0.78) indicating that treatment does not affect visual acuity. The open field test for duration of grooming (A), pausing (B) and walking (C) confirms that AZD-103 does not affect activity levels in the TgCRND8 mice or in their non-Tg littermates.

FIG. 8. Dot blot analyses of soluble oligomeric Aβ in AZD-102 and AZD-103 treated and untreated TgCRND8 mice (A,B). Soluble proteins isolated from all four and six month old untreated (solid bars) and treated (hatched bars) TgCRND8 mice from the prophylactic study (n=8-10 per experimental arm), and from the five month old treatment groups, untreated (solid bars) and treated (hatched bars) (n=8-10 per experimental arm) were applied to nitrocellulose and probed with oligomer-specific antibody followed by re-probing with 6E10. Synaptophysin reactivity was increased after AZD-103 treatment in both the prophylactic and treatment paradigms in the CA1 region (C) (n=3, for each experimental arm). In contrast, synaptophysin reactivity was unchanged in the TgTauP301L mice after treatment (n=3 for treated and untreated).

FIG. 9. In vitro γ-secretase assays in HEK293 cells transfected with human APPswe. After swAPP stable HEK293 cells were untreated or treated with AZD-103 or 10 nM compound E, cell membranes were used for APP-FL and —CTF detection and for e-stubs generation in vitro assays at 37° C. for 1 hr. Lanes 1&2.90 mg/ml AZD-103 treatment; lanes 3&4 900 mg/ml AZD-103 treatment; lanes 5&6 compound E treated and lanes 7&8 untreated HEK293 APPswe cells.

FIG. 10. Cyclohexanehexols improve behavior in TgCRND8 mice. Spatial reference memory version of the Morris water maze test in TgCRND8 mice (n=8-10 per treatment arm) was used as a measure of cognition. At 4 months of age, nontreated TgCRND8 mice showed cognitive impairment relative to mice treated with epi-cyclohexanehexol (a) and scyllocyclohexanehexol (b; F2,26=3.99, P=0.03). Epi-cyclohexanehexol-treated mice (a) were significantly different from treated and untreated nontransgenic mice (F1,18=11.7, P=0.004), whereas scyllo-cyclohexanehexol-treated mice (b) approached that of nontransgenic mice (F1,17=2.89, P=0.97). At 6 months of age, nontreated TgCRND8 mice showed cognitive impairment relative to nontransgenic control mice (F1,30=31.16, P<0.001) and mice treated with epi-cyclohexanehexol (c) or scyllocyclohexanehexol (d; F2,36=4.1, P<0.02). The performance of both epi-cyclohexanehexol—treated TgCRND8 mice (F1,21=2.35, P=0.14; c) and scyllo-cyclohexanehexol-treated TgCRND8 mice approached that of nontransgenic littermates (F1,22=3.26, P=0.44; d). Nontransgenic littermate behavior was not affected by treatment with either epi-(c) or scyllo-cyclohexanehexol (d; F2,37=0.83, P=0.45). Vertical bars represent s.e.m.

FIG. 11. Cyclohexanehexols improve pathological characteristics in TgCRND8 mice. TgCRND8 mice have a considerable vascular Aβ burden that is associated with small and medium-sized vessels (a). Scyllocyclohexanehexol treatment significantly decreased the total vascular load in comparison to untreated and epi-inositol—treated TgCRND8 mice (a). TgCRND8 mice showed an astrogliotic response to increased Aβ levels, and treatment with epi-cyclohexanehexol decreased the percent brain area covered in astrogliosis at both 4 and 6 months of age (b). Scyllo-cyclohexanehexol treatment decreased the astrogliotic response to a greater extent than epi-cyclohexanehexol at both ages but had no effect in disease stage transgenic Tau (P301L) 23027 mice (b). Similarly, microgliosis has been correlated with plaque burden in the TgCRND8 mice (c). Treatment with both epi- and to a greater extent with scyllo-cyclohexanehexol decreased the percent brain area covered with microgliosis (c). Kaplan-Meier cumulative survival plot shows the increased survival of TgCRND8 mice after treatment with scyllo-cyclohexanehexol (P=0.02) in comparison to untreated TgCRND8 mice (d). Epi-cyclohexanehexol did not significantly improve survival. Synaptophysin immunoreactivity was used as a measure of synaptic density and was increased after scyllo-cyclohexanehexol treatment in both the prophylactic and treatment paradigms in the CA1 region (e). In contrast, synaptophysin reactivity was unchanged in TgTau (P301L) mice after treatment (e). *P<0.05, **P<0.001 by ANOVA.

FIG. 12. Spatial reference memory test was performed in 6-month-old mice after 28 d of treatment, beginning at 5 months of age (n=10 mice per treatment arm). The performance of epi-cyclohexanehexol-treated TgCRND8 mice was not different from untreated TgCRND8 littermates (P=0.27; a) and remained impaired with respect to nontransgenic littermates (F1,14=11.7, P=0.004). In contrast, scyllo-cyclohexanehexol-treated TgCRND8 mice were significantly better than untreated TgCRND8 littermates (P=0.01; b) and were indistinguishable from nontransgenic littermates (F1,13=2.9, P=0.11). The probe trial, using annulus-crossing index, showed that scyllo-cyclohexanehexol-treated TgCRND8 mice were not statistically different from nontransgenic littermates (P=0.64; c). nTg, nontransgenic mice; Tg, TgCRND8 mice. Vertical bars represent s.e.m.

FIG. 13. Dot-blot analyses of soluble oligomeric Aβ in TgCRND8 left untreated or treated with epi-cyclohexanehexol (a) or scyllo-cyclohexanehexol (b). Soluble proteins isolated from untreated (black bars) and treated (gray bars) TgCRND8 mice from the 4- and 6-month prophylactic study and from the 5-month-old treatment group (n=8-10 per experimental arm) were probed with oligomer-specific antibody followed by re-probing with 6E10 (c). Synthetic Aβ42, monomeric Aβ42 and fibrillar Aβ42 were used as negative controls and aggregated Aβ42 was used as a positive control for the oligomer-specific antibody 6E10 recognizes all Aβ species and was used as an Ab positive control. Westernblot analyses of soluble fractions from 4-month-old TgCRND8 mice untreated (n=4) or treated (n=4) with scyllo-cyclohexanehexol showed a decrease in high-molecular-weight Aβ species and a subsequent increase in smaller oligomers (d). The highmolecular-weight Aβ species does not comigrate with holo-APP, as reprobing the same blot with 22C11 recognizing an N-terminal epitope in the APP ectoderm identifies a faster migrating doublet (d). The gel was reprobed with GAPDH-specific antibody as a loading control. Quantification of changes in high-molecular-weight Ab oligomer, trimer and monomer bands using densitometry confirmed the western blot analyses (e).

FIG. 14. Dose-dependent effects of scyllo-cyclohexanehexol on 4-month-old TgCRND8 mice. Four-month-old TgCRND8 mice with signs of clinical disease were administered scyllo-cyclohexanehexol orally for 1 month (n=8-9 per group). Outcome measures indicated improvement in cognitive deficits using the Morris water maze test of spatial memory (a), plaque count using 6F3D immunostaining and image analyses (b), and soluble Ab oligomers using oligomerspecific antibody in a dot-blot assay (c, d). To show that the dose-response effect is not specific to younger mice, 5-month-old TgCRND8 mice were gavaged with 0-30 mg/kg/d scyllo-cyclohexanehexol. Brain Aβ42 levels were examined and a dose-dependent decrease in both soluble and insoluble Aβ42 levels was detected (e,f).

FIG. 15. Cyclohexanehexol stereoisomer structures. The positioning of the hydroxyl groups on the ring structure of myo (1), epi-(2) and scyllo-cyclohexanehexol (3) are shown.

FIG. 16. At six months of age, the plaque burden and astrogliosis in TgCRND8 mice untreated, epi- and scyllo-cyclohexanehexol treated mice were examined. Control animals have a high plaque load and astrogliosis in the hippocampus (a) and cerebral cortex (b). Higher magnification demonstrates that astrocytic activation is not only associated with plaque load (c). Epi-cyclohexanehexol treatment has a modest effect on amyloid burden with a decrease in astrogliosis (d, e, f). Scyllo-cyclohexanehexol treatment significantly decreased amyloid burden and gliosis (g.h.i). Higher magnification illustrates the smaller mean plaque size in scyllo-cyclohexanehexol treated mice (i). Astrocytes labeled with anti-GFAP antibody (red) and plaque burden identified with anti-Aβ antibody (brown). Scale Bar 300 μm (a, b, d, e, g, h) and 62.5 μm (c, f, j).

FIG. 17. Spatial reference memory version of the Morris Water Maze test in six month old TgCRND8 mice non-treated or treated with mannitol. Mannitol treated TgCRND8 mice (dashed line) were not significantly different from untreated TgCRND8 mice (solid line: P=0.89; a). The performance of mannitol treated TgCRND8 mice (dashed line) was significantly different from mannitol treated non-Tg littermates (solid line: P=0.05; b). Vertical Bars represent SEM. Plaque burden was analysed at six months of age by quantitative image analyses (c). Mannitol treated TgCRND8 mice were indistinguishable from untreated TgCRND8 mice when plaque count was used as a measure of total plaque burden (P=0.87). Vertical bars represent SEM. Kaplan-Meier Cumulative survival plots for TgCRND8 mice treated and untreated with mannitol (d). The two cohorts of animals, n=35 per group, were not significantly different as determined by the Tarone-Ware statistical test, P=0.87.

FIG. 18. A cue test was performed at the end of the spatial memory version of the Morris Water Test. A flag was placed on the platform and the path length required to reach the platform is comparable for all treatment groups (P=0.78) indicating that treatment does not affect visual acuity (a). The open field test for duration of grooming (b), pausing (c) and walking (d) confirms that scyllo-cyclohexanehexol does not affect activity levels in the TgCRND8 mice or in their non-Tg littermates.

### DETAILED DESCRIPTION OF EMBODIMENTS

### Glossary

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition containing “a compound” includes a mixture of two or more compounds.

The terms “administering” and “administration” refer to the process by which a therapeutically effective amount of a compound or composition contemplated herein is delivered to a subject for prevention and/or treatment purposes. Compositions are administered in accordance with good medical practices taking into account the subject's clinical condition, the site and method of administration, dosage, patient age, sex, body weight, and other factors known to physicians.

The term “treating” refers to reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a compound or composition of the present invention to a subject that is not at the time of administration afflicted with the disease. “Preventing” also refers to preventing the recurrence of a disease or of one or more symptoms associated with such disease. The terms “treatment” and “therapeutically,” refer to the act of treating, as “treating” is defined above.

The terms “subject”, “individual”, or “patient” are used interchangeably herein and refer to an animal including a warm-blooded animal such as a mammal, which is afflicted with or suspected of having or being pre-disposed to a disorder and/or disease disclosed herein. Mammal includes without limitation any members of the Mammalia. In aspects of the invention, the terms refer to a human. The terms also include domestic animals bred for food or as pets, including horses, cows, sheep, poultry, fish, pigs, cats, dogs, and zoo animals, goats, apes (e.g. gorilla or chimpanzee), and rodents such as rats and mice. Typical subjects for treatment include persons susceptible to, suffering from or that have suffered an amyloid-related disease. A subject may or may not have a genetic predisposition for a disorder and/or disease disclosed herein such as Alzheimer's disease. In some aspects, a subject shows signs of cognitive deficits and amyloid plaque neuropathology. In embodiments of the invention the subjects are suspectible to, or suffer from Alzheimer's disease.

As utilized herein, the term “healthy subject” means a subject, in particular a mammal, having no disease, in particular no diagnosed disease, disorder, infirmity, or ailment known to impair or otherwise diminish memory.

The term “pharmaceutically acceptable carrier(s), excipient(s), or vehicle(s)” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc include but are not limited to saline, buffered saline, dextrose, water, glycerol, ethanol, and combinations thereof. The use of such media and agents for an active substance is well known in the art.

As used herein “nutraceutically acceptable derivative” refers to a derivative or substitute for the stated chemical species that operates in a similar manner to produce the intended effect, and is structurally similar and physiologically compatible. Examples of substitutes include without limitation salts, esters, hydrates, or complexes of the stated chemical. The substitute could also be a precursor or prodrug to the stated chemical, which subsequently undergoes a reaction in vivo to yield the stated chemical or a substitute thereof.

The term “pure” in general means better than 90%, 92%, 95%, 97%, 98% or 99% pure, and “substantially pure” means a compound synthesized such that the compound, as made as available for consideration into a composition or therapeutic dosage of the invention, has only those impurities that can not readily nor reasonably be removed by conventional purification processes.

“Pharmaceutically acceptable salt(s),” means a salt that is pharmaceutically acceptable and has the desired pharmacological properties. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Suitable salts include salts that may be formed where acidic protons in the compounds are capable of reacting with inorganic or organic bases. Suitable inorganic salts include those formed with alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloride and hydrobromic acids) and organic acids (e.g. acetic acid, citric acid, maleic acid, and the alkane- and arene-sulfonic acids such as methanesulfonic acid and benezenesulfonic acid). When there are two acidic groups present, a pharmaceutically acceptable salt may be a mono-acid-mono-salt or a di-salt; and similarly where there are more than two acidic groups present, some or all of such groups can be salified.

A “combination treatment” means that the active ingredients are administered concurrently to a patient being treated. When administered in combination each component may be administered at the same time, or sequentially in any order at different points in time. Therefore, each component may be administered separately, but sufficiently close in time to provide the desired effect, in particular a beneficial, additive, or synergistic effect. The first compound may be administered in a regimen that additionally comprises treatment with the second compound. In aspects the terms refer to the administration of a scyllo-inositol compound and a second therapeutic agent optionally within one year, including separate administration of medicaments each containing one of the compounds as well as simultaneous administration whether or not the compounds are combined in one formulation or whether they are in separate formulations.

“Detectable substance” includes without limitation radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, or epitope tags). In some embodiments, labels are attached via spacer arms of various lengths to reduce potential steric hindrance.

A “beneficial effect” refers to an effect of a compound of the invention or composition thereof in certain aspects of the invention, including favorable pharmacological and/or therapeutic effects, and/or improved biological activity. In aspects of the invention, the beneficial effects include without limitation improved cognitive function, reduced vascular load, reduced astrogliosis, reduced amyloid burden, reduced microgliosis, and/or improved survival. In an aspect, a beneficial effect is a favourable characteristic of a composition/formulation of the invention includes enhanced stability, a longer half life, and/or enhanced uptake and transport across the blood brain barrier. In some aspects, a beneficial effect of a composition of the invention is rapid brain penetrance, in particular brain penetrance within 1-6, 1-5, 1-4, 1-3 or 1-2 hours of administration.

The beneficial effect may be a statistically significant effect in terms of statistical analysis of an effect of a scyllo-inositol compound versus the effects without the compound. In an aspect, the beneficial effect is one or more of improved cognitive function, reduced vascular load, reduced astrogliosis, reduced amyloid burden, reduced microgliosis, and/or improved survival. “Statistically significant” or “significantly different” effects or levels may represent levels that are higher or lower than a standard. In embodiments of the invention, the difference may be 1.5, 2, 3, 4, 5, 6, 7, 8, 9, 10, 15, 20, 25 or 50 times higher or lower compared with the effect obtained without a scyllo-inositol compound.

“Therapeutically effective amount” relates to the amount or dose of an active compound or composition of the invention that will provide or lead to one or more desired beneficial effects, in particular, one or more sustained beneficial effects. A therapeutically effective amount of a substance can vary according to factors such as the disease state, age, sex, and weight of the individual, and the ability of the substance to elicit a desired response in the individual. A dosage regimen may be adjusted to provide the optimum therapeutic response (e.g. one or more beneficial effect, in particular a sustained beneficial effect). For example, several divided doses may be administered daily or the dose may be proportionally reduced as indicated by the exigencies of the therapeutic situation.

“A scyllo-inositol compound” is understood to refer to any compound, which fully or partially, directly or indirectly, provides one or more beneficial effects described herein. A scyllo-inositol compound that can be used in the invention has the base structure of the formula Ia or Ib:

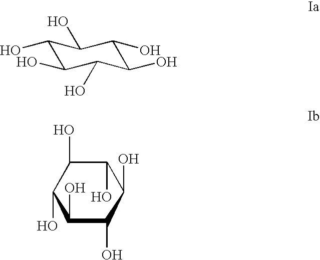

A scyllo-inositol compound includes a functional derivative of a compound of the formula Ia or Ib. A “functional derivative” refers to a compound that possesses a biological activity (either functional or structural) that is substantially similar to the biological activity of scyllo-inositol of the formula Ia or Ib. The term “functional derivative” is intended to include “variants” “analogs” or “chemical derivatives” of scyllo-inositol. The term “variant” is meant to refer to a molecule substantially similar in structure and function to scyllo-inositol or a part thereof. A molecule is “substantially similar” to scyllo-inositol if both molecules have substantially similar structures or if both molecules possess similar biological activity. The term “analog” refers to a molecule substantially similar in function to a scyllo-inositol molecule. The term “chemical derivative” describes a molecule that contains additional chemical moieties which are not normally a part of the base molecule.

A scyllo-inositol compound of the invention includes crystalline forms of the compound which may exist as polymorphs. Solvates of the compounds formed with water or common organic solvents are also intended to be encompassed within this invention. In addition, hydrate forms of scyllo-inositol compounds and their salts, are included within this invention.

A scyllo-inositol compound includes a compound of the formula Ia or Ib wherein one, two or three hydroxyl groups are replaced by substituents, in particular univalent substituents, with retention of configuration. Suitable substituents include without limitation hydrogen, alkyl, acyl, alkenyl, alkoxy, ═O, cycloalkyl, halogen, —NHR1 wherein R1 is hydrogen, acyl, alkyl or —R2R3 wherein R2 and R3 are the same or different and represent acyl or alkyl; —PO3H2; —SR4 wherein R4 is hydrogen, alkyl, or —O3H; and —OR3 wherein R3 is hydrogen, alkyl, or —SO3H. In aspects of the invention, a scyllo-inositol compound does not include scyllo-inositol substituted with one or more phosphate group.

Particular aspects of the invention utilize scyllo-inositol compounds of the formula Ia or Ib wherein one or more of the hydroxyl groups are replaced with alkyl, acyl, alkoxy, alkenyl, —NHR1 wherein R1 is hydrogen, acyl, alkyl or —R2R3 wherein R2 and R3 are the same or different and represent acyl or alkyl; —SR4 wherein R4 is hydrogen, alkyl, or —O3H; and —OR3 wherein R3 is hydrogen, alkyl, or —SO3H, more particularly —SR4 wherein R4 is hydrogen, alkyl, or —O3H or —SO3H.

Particular aspects of the invention utilize scyllo-inositol compounds of the formula Ia or Ib wherein one or more of the hydroxyl groups are replaced with C1-C6alkyl, C2-C6alkenyl, C1-C6alkoxy, C3-C10cycloalkyl, C1-C6 acyl, —NH2, —NHR1, —NR2R3, halo, haloalkyl, haloalkoxy, hydroxyalkyl, or oxo.

Particular aspects of the invention utilize scyllo-inositol compounds of the formula Ia or Ib wherein one or more of the hydroxyl groups is replaced with C1-C6 alkyl, C2-C6 alkenyl, C1-C6alkoxy, C1-C6 acyl, —NH2, halo, or oxo.

In embodiments of the invention, a scyllo-inositol compound of the formula Ia or Ib is employed wherein one or two hydroxyl groups are replaced by ═O. In particular embodiments of the invention one hydroxyl group is replace by ═O, more particularly the hydroxyl group at position 2 is replaced by ═O.

In embodiments, scyllo-cyclohexanehexol (i.e., scyllo-inositol), in particular pure or substantially pure scyllo-cyclohexanehexol, is used in the compositions, methods and uses disclosed herein.

“Alkyl” refers to monovalent alkyl groups preferably having from 1 to 20 or 1 to 10 carbon atoms, preferably from about 1 to 10, 1 to 8, 3 to 8, 1 to 6, or 1 to 3, more preferably about 3 to 6 carbon atoms. This term is exemplified by groups such as methyl, ethyl, n-propyl, iso-propyl, n-butyl, iso-butyl, n-hexyl, isopropyl, isobutyl, isopentyl, amyl, sec-butyl, tert-butyl, tert-pentyl, n-heptyl, n-octyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain embodiments of the invention an alkyl radical is a C1-C6 lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl group can be a substituted alkyl.

“Substituted alkyl” refers to an alkyl group, preferably of from 1 to 10 carbon atoms, having from 1 to 5 substituents, and preferably 1 to 3 substituents, for example, alkyl, alkoxy in particular lower alkoxy, cycloalkyl, acyl, amino, substituted amino, cyano, halo, hydroxyl, carboxyl, substituted carboxyl, carboxylalkyl, keto, thioketo, thiol, thioalkoxy, aryl, hydroxyamino, alkoxyamino, nitro, sulfonyl, sulfenyl, sulfinyl, sulfate, or sulfoxide.

“Alkenyl” refers to alkenyl groups preferably having from 2 to 10 carbon atoms and more preferably 3 to 8 carbon atoms and having at least 1 and preferably from 1-2 sites of alkenyl unsaturation. Alkenyl radicals may preferably contain from about 3 to 6 or 2 to 6 carbon atoms. Examples of suitable alkenyl radicals include ethenyl, propenyl such as prop-1-en-1-yl, prop-1-en-2-yl, prop-2-en-1-yl (allyl), prop-2-en-2-yl, buten-1-yl, but-1-en-2-yl, 2-methyl-prop-1-en-1-yl, but-2-en-1-yl, but-2-en-2-yl, buta-1,3-dien-1-yl, buta-1,3-dien-2-yl, hexen-1-yl, 3-hydroxyhexen-1-yl, hepten-1-yl, and octen-1-yl, and the like. Preferred alkenyl groups include ethenyl (—CH═CH2), n-propenyl (—CH2CH═CH2), iso-propenyl (—C(CH3)═CH2), and the like.

“Substituted alkenyl” refers to an alkenyl group as defined above having from 1 to 3 substituents, for example, alkyl, alkoxy in particular lower alkoxy, cycloalkyl, acyl, amino, substituted amino, cyano, halo, hydroxyl, carboxyl, substituted carboxyl, carboxylalkyl, keto, thioketo, thiol, thioalkoxy, aryl, hydroxyamino, alkoxyamino, nitro, sulfonyl, sulfenyl, sulfinyl, sulfate, or sulfoxide

“Acyl” refers to the groups alkyl-C(O)—, substituted alkyl-C(O)—, cycloalkyl-C(O)—, substituted cycloalkyl-C(O)—, aryl-C(O)—, heteroaryl-C(O)— and heterocyclic-C(O)— where alkyl, substituted alkyl, cycloalkyl, substituted cycloalkyl, aryl, substituted aryl, heteroaryl and heterocyclic are as defined herein. An acyl group may be substituted with for example a group disclosed herein for alkyl. Illustrative examples of “acyl” radicals are formyl, acetyl, 2-chloroacetyl, 2-bromacetyl, benzoyl, trifluoroacetyl, phthaloyl, malonyl, nicotinyl, and the like.

“Alkoxy” refers to a linear or branched oxy-containing radical having an alkyl portion of one to about ten carbon atoms, such as a methoxy radical, which may be substituted. Particular alkoxy radicals are “lower alkoxy” radicals having about 1 to 6, 1 to 4 or 1 to 3 carbon atoms. An alkoxy having about 1-6 carbon atoms includes a C1-C6 alkyl-O— radical wherein C1-C6 alkyl has the meaning set out herein. Illustrative examples of alkoxy radicals include without limitation methoxy, ethoxy, propoxy, butoxy, isopropoxy and tert-butoxy. An “alkoxy” radical may optionally be further substituted with one or more substitutents disclosed herein including alkyl atoms (in particular lower alkyl) to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropoxy) and “haloalkoxyalkyl” radicals (e.g. fluoromethoxymethyl, chloromethoxyethyl, trifluoromethoxymethyl, difluoromethoxyethyl, and trifluoroethoxymethyl).

“Aryl” refers to an unsaturated aromatic carbocyclic group of from 6 to 14 carbon atoms having a single ring (e.g., phenyl) or multiple condensed (fused) rings (e.g., naphthyl or anthryl). Preferred aryls include phenyl, naphthyl and the like. In aspects of the invention an aryl radical has 4 to 24 carbon atoms, in particular 4 to 10, 4 to 8, or 4 to 6 carbon atoms. The term “aryl” includes without limitation aromatic radicals such as phenyl, naphthyl, indenyl, benzocyclooctenyl, benzocycloheptenyl, pentalenyl, azulenyl, tetrahydronaphthyl, indanyl, biphenyl, acephthylenyl, fluorenyl, phenalenyl, phenanthrenyl, and anthracenyl, preferably phenyl. An aryl group may be a substituted aryl group which may include an aryl group as defined herein having from 1 to 8, 1 to 6, 1 to 4, or 1 to 3 substituents, for example, alkyl, alkoxy, cycloalkyl, acyl, amino, cyano, halogen, hydroxyl, carboxyl, carboxylalkyl, keto, thioketo, thiol, thioalkoxy, aryl, hydroxyamino, alkoxyamino, and nitro. Examples of substituted aryl radicals include benzyl, chlorobenyzl, and amino benzyl.

“Cycloalkyl” refers to cyclic alkyl groups of from 3 to 16, 3 to 15 or 3 to 12 carbon atoms having a single cyclic ring or multiple condensed rings.

In aspects of the invention a cycloalkyl comprises one, two, three, or fourrings wherein such rings may be attached in a pendant manner or may be fused, in particular cyclopropyl, cyclobutyl, cyclopentyl, cyclohexyl, cycloheptyl, cyclooctyl, cyclononyl, adamantyl, and the like. In certain aspects of the invention the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl, cycloheptyl and cyclooctyl. A cycloalkyl group includes multiple ring structures such as adamantanyl, and the like. The term “cycloalkyl” also embraces radicals where cycloalkyl radicals are fused with aryl radicals or heterocyclyl radicals. A cycloalkyl radical may be optionally substituted with groups as disclosed herein.

“Substituted cycloalkyl” refers to cycloalkyl groups having from 1 to 5 (in particular 1 to 3) substituents including without limitation alkoxy, cycloalkyl, substituted cycloalkyl, cycloalkenyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, aryl, aryloxy, heteroaryl, heteroaryloxy, heterocyclic, heterocyclooxy, hydroxyamino, alkoxyamino, and nitro.

“Halogen” refers to fluoro, chloro, bromo and iodo and preferably is either fluoro or chloro.

“Heteroaryl” refers to an aromatic group of from 1 to 15 carbon atoms and 1 to 4 heteroatoms selected from oxygen, nitrogen and sulfur within at least one ring (if there is more than one ring). Such heteroaryl groups can be optionally substituted with 1 to 5 substituents, for example, acyloxy, hydroxy, acyl, alkyl, alkoxy, alkenyl, alkynyl, substituted alkyl, substituted alkenyl, substituted alkynyl, amino, substituted amino, aminoacyl, acylamino, alkaryl, aryl, aryloxy, azido, carboxyl, carboxylalkyl, cyano, halo, nitro, heteroaryl, heterocyclic, aminoacyloxy, oxyacylamino, thioalkoxy, substituted thioalkoxy, thioaryloxy, and thioheteroaryloxy. Such heteroaryl groups can have a single ring (e.g., pyridyl or furyl) or multiple condensed rings (e.g., indolizinyl or benzothienyl).

“Heterocycle” or “heterocyclic” refers to a monovalent saturated or unsaturated group having a single ring or multiple condensed rings, from 1 to 15 carbon atoms and from 1 to 4 hetero atoms selected from nitrogen, sulfur or oxygen within the ring. Heterocyclic groups can have a single ring or multiple condensed rings. Heterocyclic groups can be optionally substituted with 1 to 5 substituents, for example, alkoxy, cycloalkyl, substituted cycloalkyl, cycloalkenyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, aryl, aryloxy, heteroaryl, heteroaryloxy, heterocyclic, heterocyclooxy, hydroxyamino, alkoxyamino, or nitro.

Examples of heterocycles and heteroaryls include, without limitation, pyrrole, furan, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

Scyllo-inositol compounds can be prepared using conventional processes or they may be obtained from commercial sources. A scyllo-inositol compound can be prepared using chemical and/or microbial processes. In aspects of the invention, a scyllo-inositol compound is produced using process steps described by M. Sarmah and Shashidhar, M., Carbohydrate Research, 2003, 338, 999-1001; Husson, C., et al, Carbohyrate Research 307 (1998) 163-165; Anderson R. and E. S. Wallis, J. American Chemical Society (US), 1948, 70:2931-2935; Weissbach, A., J Org Chem (US), 1958, 23:329-330; Chung, S. K. et al., Bioorg Med. Chem. 1999, 7(11):2577-89; or Kiely D. E., and Fletcher, H. G., J. American Chemical Society (US) 1968, 90:3289-3290; described in JP09-140388, DE 3,405,663, JP04-126075, JP05-192163, or WO06109479, or described in WO0503577, US20060240534, EP1674578, JP9140388, JP09140388, JP02-184912, JP03-102492 (Hokko Chemical Industries). In particular aspects of the compositions and methods of the invention, a scyllo-inositol compound is prepared using the chemical process steps described in Husson, C., et al, Carbohyrate Research 307 (1998) 163-165. In other aspects of the compositions and methods of the invention, the scyllo-inositol compound is prepared using microbial process steps described in WO05035774 (EP1674578 and US20060240534) (Hokko Chemical Industries). Derivatives may be produced by introducing substituents into a scyllo-inositol using methods well known to a person of ordinary skill in the art

A scyllo-inositol compound may additionally comprise a carrier, including without limitation one or more of a polymer, carbohydrate, peptide or derivative thereof. A carrier may be substituted with substituents described herein including without limitation one or more alkyl, amino, nitro, halogen, thiol, thioalkyl, sulfate, sulfonyl, sulfenyl, sulfinyl, sulfoxide, hydroxyl groups. A carrier can be directly or indirectly covalently attached to a compound of the invention. In aspects of the invention the carrier is an amino acid including alanine, glycine, proline, methionine, serine, threonine, or asparagine. In other aspects the carrier is a peptide including alanyl-alanyl, prolyl-methionyl, or glycyl-glycyl.

A carrier also includes a molecule that targets a compound of the invention to a particular tissue or organ. In particular, a carrier may facilitate or enhance transport of a compound of the invention to the brain by either active or passive transport.

A “polymer” as used herein refers to molecules comprising two or more monomer subunits that may be identical repeating subunits or different repeating subunits. A monomer generally comprises a simple structure, low-molecular weight molecule containing carbon. Polymers can be optionally substituted. Examples of polymers which can be used in the present invention are vinyl, acryl, styrene, carbohydrate derived polymers, polyethylene glycol (PEG), polyoxyethylene, polymethylene glycol, poly-trimethylene glycols, polyvinylpyrrolidone, polyoxyethylene-polyoxypropylene block polymers, and copolymers, salts, and derivatives thereof. In particular aspects of the invention, the polymer is poly(2-acrylamido-2-methyl-1-propanesulfonic acid); poly(2-acrylamido-2-methyl,-1-propanesulfonic acid-coacrylonitrile, poly(2-acrylamido-2-methyl-1-propanesulfonic acid-co-styrene), poly(vinylsulfonic acid); poly(sodium 4-styrenesulfonic acid); and sulfates and sulfonates derived therefrom; poly(acrylic acid), poly(methylacrylate), poly(methyl methacrylate), and poly(vinyl alcohol).

A “carbohydrate” as used herein refers to a polyhydroxyaldehyde, or polyhydroxyketone and derivatives thereof. The simplest carbohydrates are monosaccharides, which are small straight-chain aldehydes and ketones with many hydroxyl groups added, usually one on each carbon except the functional group. Examples of monosaccharides include erythrose, arabinose, allose, altrose, glucose, mannose, threose, xylose, gulose, idose, galactose, talose, aldohexose, fructose, ketohexose, ribose, and aldopentose. Other carbohydrates are composed of monosaccharide units, including disaccharides, oligosaccharides, or polysaccharides, depending on the number of monosaccharide units. Disaccharides are composed of two monosaccharide units joined by a covalent glycosidic bond. Examples of disaccharides are sucrose, lactose, and maltose. Oligosaccharides and polysaccharides, are composed of longer chains of monosaccharide units bound together by glycosidic bonds. Oligosaccharides generally contain between 3 and 9 monosaccharide units and polysaccharides contain greater than 10 monosaccharide units. A carbohydrate group may be substituted at one two, three or four positions, other than the position of linkage to a compound of the formula Ia or Ib. For example, a carbohydrate may be substituted with one or more alkyl, amino, nitro, halo, thiol, carboxyl, or hydroxyl groups, which are optionally substituted. Illustrative substituted carbohydrates are glucosamine or galactosamine.

In aspects of the invention, the carbohydrate is a sugar, in particular a hexose or pentose and may be an aldose or a ketose. A sugar may be a member of the D or L series and can include amino sugars, deoxy sugars, and their uronic acid derivatives. In embodiments of the invention where the carbohydrate is a hexose, the hexose is selected from the group consisting of glucose, galactose, or mannose, or substituted hexose sugar residues such as an amino sugar residue such as hexosamine, galactosamine, glucosamine, in particular D-glucosamine (2-amino-2-doexy-D-glucose) or D-galactosamine (2-amino-2-deoxy-D-galactose). Suitable pentose sugars include arabinose, fucose, and ribose.

The term “carbohydrate” also includes glycoproteins such as lectins (e.g. concanavalin A, wheat germ agglutinin, peanutagglutinin, seromucoid, and orosomucoid) and glycolipids such as cerebroside and ganglioside.

A “peptide” for use as a carrier in the practice of the present invention includes one, two, three, four, or five or more amino acids covalently linked through a peptide bond. A peptide can comprise one or more naturally occurring amino acids, and analogs, derivatives, and congeners thereof. A peptide can be modified to increase its stability, bioavailability, solubility, etc. “Peptide analogue” and “peptide derivative” as used herein include molecules which mimic the chemical structure of a peptide and retain the functional properties of the peptide. In aspects of the invention the carrier is an amino acid such as alanine, glycine, proline, methionine, serine, threonine, histidine, or asparagine. In other aspects the carrier is a peptide such as alanyl-alanyl, prolyl-methionyl, or glycyl-glycyl. In still other aspects, the carrier is a polypeptide such as albumin, antitrypsin, macroglobulin, haptoglobin, ceruloplasm, transferrin, α- or β-lipoprotein, β- or γ-globulin or fibrinogen.

Approaches to designing peptide analogues, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

Examples of peptide analogues, derivatives and peptidomimetics include peptides substituted with one or more benzodiazepine molecules (see e.g., James, G. L. et al. (1993) Science 260:1937-1942), peptides with methylated amide linkages and “retro-inverso” peptides (see U.S. Pat. No. 4,522,752 by Sisto).

Examples of peptide derivatives include peptides in which an amino acid side chain, the peptide backbone, or the amino- or carboxy-terminus has been derivatized (e.g., peptidic compounds with methylated amide linkages).

The term mimetic, and in particular, peptidomimetic, is intended to include isosteres. The term “isostere” refers to a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the alpha-carbon, amide carbonyl, complete replacement of the amide bond, extensions, deletions or backbone crosslinks. Other examples of isosteres include peptides substituted with one or more benzodiazepine molecules (see e.g., James, G. L. et al. (1993) Science 260:1937-1942)

Other possible modifications include an N-alkyl (or aryl) substitution ([CONR]), backbone crosslinking to construct lactams and other cyclic structures, substitution of all D-amino acids for all L-amino acids within the compound (“inverso” compounds) or retro-inverso amino acid incorporation ([NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides.

A peptide can be attached to a compound of the invention through a functional group on the side chain of certain amino acids (e.g. serine) or other suitable functional groups. In an embodiment of the invention the carrier may comprise four or more amino acids with groups attached to three or more of the amino acids through functional groups on side chains. In another embodiment, the carrier is one amino acid, in particular a sulfonate derivative of an amino acid, for example cysteic acid.

“An amyloid-related disease” includes a disease associated with the accumulation of amyloid fibrils, which can either be restricted to one organ, i.e. “localized amyloidosis”, or spread to several organs, which is denoted “systemic amyloidosis”. An amyloid-related disease includes amyloid diseases or amyloidoses. In aspects of the invention, the term include conditions associated with the formation, deposition, accumulation, or persistence of amyloid or amyloid fibrils, comprising an amyloid protein comprising or selected from the group consisting of Aβ amyloid, AA amyloid, AL amyloid, IAPP amyloid, PrP amyloid, α2-microglobulin amyloid, transthyretin, prealbumin, and procalcitonin, especially Aβ amyloid and IAPP amyloid. A disorder and/or disease may be a condition where it is desirable to dissociate abnormally aggregated proteins and/or dissolve or disrupt pre-formed or pre-deposited amyloid or amyloid fibril.

In certain aspects of the invention the disease is an amyloidosis. “Amyloidosis” refers to a diverse group of diseases of acquired or hereditary origin and characterized by the accumulation of one of several different types of protein fibrils with similar properties called amyloid. Amyloid can accumulate in a single organ or be dispersed throughout the body. The disease can cause serious problems in the affected areas, which may include the heart, brain, kidneys and digestive tract. The fibrillar composition of amyloid deposits is an identifying characteristic for various amyloid diseases. Intracerebral and cerebrovascular deposits composed primarily of fibrils of beta amyloid peptide (β-AP) are characteristic of Alzheimer's disease (both familial and sporadic forms); islet amyloid protein peptide (IAPP; amylin) is characteristic of the fibrils in pancreatic islet cell amyloid deposits associated with type II diabetes; and, β-2-microglobulin is a major component of amyloid deposits which form as a consequence of long term hemodialysis treatment. Prion-associated diseases, such as Creutzfeld-Jacob disease, scrapie, bovine spongiform encephalitis, and the like are characterized by the accumulation of a protease-resistant form of a prion protein (designated as AScr ro PrP-27).

Certain disorders are considered to be primary amyloidoses, in which there is no evidence for preexisting or coexisting disease. Primary amyloidoses are typically characterized by the presence of “amyloid light chain-type” (AL-type) protein fibrils. In secondary amyloidosis there is an underlying chronic inflammatory or infectious disease state (e.g., rheumatoid arthritis, juvenile chronic arthritis, ankylosing spondylitis, psoriasis, Reiter's syndrome, Adult Still's disease, Behcet's Syndrome, Crohn's disease, chronic microbial infections such as osteomyelitis, tuberculosis, and leprosy, malignant neoplasms such as Hodgkin's lymphoma, renal carcinoma, carcinomas of the gut, lung, and urogenital tract, basel cell carcinoma, and hairy cell carcinoma). Secondary amyloidosis is characterized by deposition of AA type fibrils derived from serum amyloid A protein (ApoSSA). Heredofamilial amyloidoses may have associated neuropathic, renal, or cardiovascular deposits of the ATTR transthyretin type, and they include other syndromes having different amyloid components (e.g., familial Mediterranean fever which is characterized by AA fibrils). Other forms of amyloidosis include local forms, characterized by focal, often tumor-like deposits that occur in isolated organs. In addition, amyloidoses are associated with aging, and are commonly characterized by plaque formation in the heart or brain. Amyloidoses includes systemic diseases such as adult-onset disabetes, complications from long-term hemodialysis and consequences of chronic inflammation or plasma cell dyscrasias.

Amyloid-related diseases that can be treated and/or prevented using the compounds, compositions and methods of the invention include without limitation, Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, nephropathy with urticaria and deafness (Muckle-Wells syndrome), amyloidosis associated with systemic inflammatory diseases, idiopathic primary amyloidosis associated with myeloma or macroglobulinemia; amyloidosis associated with immunocyte dyscrasia; monoclonal gammopathy; occult dyscrasia; local nodular amyloidosis associated with chronic inflammatory diseases; amyloidosis associated with several immunocyte dyscrasias; familial amyloid polyneuropathy; hereditary cerebral hemorrhage with amyloidosis Alzheimer's disease and other neurodegenerative diseases, amyloidosis associated with chronic hemodialysis, diabetes type II, insulinoma, the amyloidosis of the prion diseases, (transmissible spongiform encephalopathies prion diseases), Creutzfeldt-Jakob disease, Gerstmann-Straussler syndrome, Kuru, and scrapie, the amyloidosis associated with carpal tunnel syndrome, senile cardiac amyloidosis, familial amyloidotic polyneuropathy, and the amyloidosis associated with endocrine tumors, especially Alzheimer's disease and type 2 diabetes.

In aspects of the invention, the amyloid-related diseases included or are selected from the group consisting of Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, the amyloidosis of the prion diseases, Creutzfeldt-Jakob disease, Gerstmann-Straussler syndrome, kuru, and scrapie, the amyloidosis associated with carpal tunnel syndrome, senile cardiac amyloidosis, familial amyloidotic polyneuropathy, and the amyloidosis associated with endocrine tumors, especially Alzheimer's disease and type 2 diabetes.

In other aspects of the invention, the amyloid-related diseases included or are selected from the group consisting of conditions of the central or peripheral nervous system or a systemic organ that result in the deposition of proteins, protein fragments, and peptides in beta-pleated sheets, fibrils, and/or aggregates or oligomers. In particular the disease is Alzheimer's disease, presenile and senile forms; amyloid angiopathy; mild cognitive impairment; Alzheimer's disease-related dementia (e.g., vascular or Alzheimer dementia); tauopathy (e.g., argyrophilic grain dementia, corticobasal degeneration, dementia pugilistica, diffuse neurofibrillary tangles with calcification, frontotemporal dementia with parkinsonism, Prion-related disease, Hallervorden-Spatz disease, myotonic dystrophy, Niemann-Pick disease type C, non-Guamanian Motor Neuron disease with neurofibrillary tangles, Pick's disease, postencephalitic parkinsonism, cerebral amyloid angiopathy, progressive subcortical gliosis, progressive supranuclear palsy, subacute sclerosing panencephalitis, and tangle only dementia), alpha-synucleinopathy (e.g., dementia with Lewy bodies, multiple system atrophy with glial cytoplasmic inclusions, Shy-Drager syndrome, spinocerebellar ataxia (e.g., DRPLA or Machado-Joseph Disease); striatonigral degeneration, olivopontocerebellar atrophy, neurodegeneration with brain iron accumulation type I, olfactory dysfunction, and amyotrophic lateral sclerosis); Parkinson's disease (e.g., familial or non-familial); Amyotrophic Lateral Sclerosis; Spastic paraplegia (e.g., associated with defective function of chaperones and/or triple A proteins); Huntington's Disease, spinocerebellar ataxia, Freidrich's Ataxia; neurodegenerative diseases associated with intracellular and/or intraneuronal aggregates of proteins with polyglutamine, polyalanine or other repeats arising from pathological expansions of tri- or tetra-nucleotide elements within corresponding genes; cerebrovascular diseases; Down's syndrome; head trauma with post-traumatic accumulation of amyloid beta peptide; Prion related disease (Creutzfeldt-Jakob disease, Gerstmann-Straussler-Scheinker disease, and variant Creutzfeldt-Jakob disease); Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy (e.g., senile amyloid polyneuropathy or systemic Amyloidosis); Inclusion Body myositis due to amyloid beta peptide; Familial and Finnish Type Amyloidosis; Systemic amyloidosis associated with multiple myeloma; Familial Mediterranean Fever; chronic infections and inflammations; and Type II Diabetes Mellitus associated with islet amyloid polypeptide (IAPP).

In aspects of the invention, in particular combination therapies, the amyloid-related disease is a neuronal disorder (e.g., Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction and dementia).

In aspects of the invention, the disease is an Amyloid Polyneuropathy including senile amyloid polyneuropathy or systemic amyloidosis.

In embodiments of the invention, the disease is Alzheimer's disease or Parkinson's disease including familial and non-familial types. In particular embodiments of the invention, the disease is Alzheimer's disease.

In embodiments of the invention, the disease is mild cognitive impairment.

In embodiments of the invention, the disease is dementia, in particular vascular, dementia with Lewy bodies, mixed dementia, Alzheimer dementia, or a secondary dementia caused by drugs, delirium, or depression. A dementia is generally characterized by loss of integrated central nervous system functions, resulting in the inability to understand simple concepts or instructions, to store and retrieve information into memory, and in behavioral and personality changes. Diagnostic features of dementia according to the DSM-IV (Diagnostic and Statistical Manual for Mental Disorders, American Psychiatric Association) include memory impairment and at least one of the following: language impairment (aphasia), lost ability to execute learned motor functions (apraxia), inability to recognize familiar objects (agnosia), or disturbances in executive functioning or decision making.

### Compositions

A scyllo-inositol compound may be formulated into a pharmaceutical composition or dietary supplement for administration to a subject. Pharmaceutical compositions of the present invention or fractions thereof typically comprise suitable pharmaceutically acceptable carriers, excipients, and vehicles selected based on the intended form of administration, and consistent with conventional pharmaceutical practices. Particular compositions of the invention can contain a scyllo-inositol compound that is pure or substantially pure.

Suitable pharmaceutical carriers, excipients, and vehicles are described in the standard text, Remington: The Science and Practice of Pharmacy, 21st Edition. University of the Sciences in Philadelphia (Editor), Mack Publishing Company. By way of example, for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

The invention provides commercially useful formulations including without limitation pills, tablets, caplets, soft and hard gelatin capsules, lozenges, sachets, cachets, vegicaps, liquids, liquid drops, elixirs, suspensions, emulsions, solutions, syrups, aerosols (as a solid or in a liquid medium) suppositories, sterile injectable solutions, and/or sterile packaged powders, which contain a scyllo-inositol compound, in particular a pure or substantially pure scyllo-compound.

A composition can be a sustained release formulation or formulated as a suppository with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g. encapsulation in liposomes, microparticles, microcapsules, and the like.

In aspects of the invention, a pharmaceutical composition is provided for oral administration of one or more scyllo-inositol compound for treatment of an amyloid related disease. In a particular aspect, a stable oral pharmaceutical composition for treatment of a disease characterized by abnormal protein folding and/or aggregation, and/or amyloid formation, deposition, accumulation, or persistence (e.g., Alzheimer's disease) is provided comprising a substantially pure scyllo-inositol compound of the formula Ia or Ib.

Formulations for parenteral administration may include aqueous solutions, syrups, aqueous or oil suspensions and emulsions with edible oil such as cottonseed oil, coconut oil, almond oil, or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

Compositions of the invention can be formulated as pharmaceutically acceptable salts as described herein.

In aspects of the invention, the compositions include without limitation at least one buffering agent or solution. Examples of buffering agents include, but are not limited to hydrochloric, hydrobromic, hydroiodic, sulfuric, phosphoric, formic, acetic, propionic, succinic, glycolic, glucoronic, maleic, furoic, citric, glutamic, benzoic, anthranilic, salicylic, phenylacetic, mandelic, embonic, pamoic, methanesulfonic, ethanesulfonic, pantothenic, benzenesulfonic, stearic, sulfanilic, algenic, galacturonic acid and mixtures thereof. Additional agents may also be included such as one or more of pregelatinized maize starch, polyvinyl pyrrolidone, hydroxypropyl methylcellulose, lactose, microcrystalline cellulose, calcium hydrogen phosphate, magnesium stearate, talc, silica, potato starch, sodium starch glycolate, sodium lauryl sulfate, sorbitol syrup, cellulose derivatives, hydrogenated edible fats, lecithin, acacia, almond oil, oily esters, ethyl alcohol, fractionated vegetable oils, methyl, propyl-p-hydroxybenzoates, sorbic acid and mixtures thereof. A buffering agent may additionally comprise one or more of dichlorodifluoromethane, trichlorofluoromethane, dichlorotetra fluoroethane, carbon dioxide, poly (N-vinyl pyrrolidone), poly (methylmethacrylate), polyactide, polyglycolide and mixtures thereof. In an embodiment, a buffering agent can be formulated as at least one medium including without limitation a suspension, solution, or emulsion. In other embodiments, a buffering agent may additionally comprise a formulatory agent including without limitation a pharmaceutically acceptable carrier, excipient, suspending agent, stabilizing agent or dispersing agent.

A composition of the invention may be sterilized by, for example, filtration through a bacteria retaining filter, addition of sterilizing agents to the composition, irradiation of the composition, or heating the composition. Alternatively, the compounds or compositions of the present invention may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the invention, such labeling would include amount, frequency, and method of administration.

A scyllo-inositol compound may be in a form suitable for administration as a dietary supplement. A supplement of the invention may optionally include inactive ingredients such as diluents or fillers, viscosity-modifying agents, preservatives, flavorings, colorants, or other additives conventional in the art. By way of example only, conventional ingredients such as beeswax, lecithin, gelatin, glycerin, caramel, and carmine may be included.

A dietary supplement composition of the invention may optionally comprise a second active ingredient. In an embodiment, the second active ingredient is pinitol or an active derivative or metabolite thereof. Pinitol can be produced from plant sources, including without limitation alfalfa, Bougainvillea leaves, chick peas, pine trees and soy beans. Pinitol is also commercially available, for example Inzitol™ (Humanetics Corporation, Min). Examples of derivatives and metabolites of pinitol include without limitation pinitol glycosides, pinitol phospholipids, esterified pinitol, lipid-bound pinitol, pinitol phosphates, pinitol phytates, and hydrolyzed pinitol such as d-chiro-inositol.

A dietary supplement may be provided as a liquid dietary supplement (e.g., a dispensable liquid) or alternatively the compositions may be formulated as granules, capsules or suppositories. The liquid supplement may include a number of suitable carriers and additives including water, glycols, oils, alcohols, flavoring agents, preservatives, coloring agents and the like. In capsule, granule or suppository form, the compositions of the present invention are formulated in admixture with a pharmaceutically acceptable carrier. In an aspect, a dietary supplement of the present invention is formulated as a beverage, but may be formulated in granule, capsule or suppository form.

A supplement may be presented in the form of a softgel which is prepared using conventional methods. A softgel typically includes a layer of gelatin encapsulating a small quantity of the supplement. A supplement may also be in the form of a liquid-filled and sealed gelatin capsule, which may be made using conventional methods.

To prepare a dietary supplement composition of the present invention in capsule, granule or suppository form, one or more compositions of the present invention may be intimately admixed with a pharmaceutically acceptable carrier according to conventional formulation techniques. For solid oral preparations such as capsules and granules, suitable carriers and additives such as starches, sugars, diluents, granulating agents, lubricants, binders, disintegrating agents and the like may be included.

According to another aspect of the invention, a kit is provided. In an aspect, the kit comprises a compound or a pharmaceutical composition of the invention. The kit can be a package which houses a container which contains a composition of the invention and also houses instructions for administering the composition to a subject.

In embodiments of the invention, a pharmaceutical pack or kit is provided comprising one or more containers filled with one or more of the ingredients of a pharmaceutical composition of the invention to provide a beneficial effect, in particular a sustained beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

### Applications

The invention is related to compositions and methods that utilize one or more scyllo-inositol compound to provide beneficial effects. In particular, the invention contemplates the use of a composition of the invention for treating an amyloid-related disease, in particular preventing, and/or ameliorating disease severity, disease symptoms, and/or periodicity of recurrence of disease. The invention also contemplates preventing and/or treating in mammals, amyloid-related diseases using the compositions or treatments of the invention. The present invention in embodiments may provide a composition comprising a compound that provides beneficial effects including greater solubility, stability, efficacy, potency, and/or utility, in particular greater solubility and stability.

In an aspect, the invention provides a method of improving memory of a healthy subject or the memory of a subject with age impaired memory by administering an effective amount of one or more scyllo-inositol compound, or a composition comprising one or more scyllo-inositol compound, and a pharmaceutically acceptable carrier, excipient, or vehicle.

In another aspect, the present invention further relates to a method for improving memory, especially short-term memory and other mental dysfunction associated with the aging process comprising administering an effective amount of one or more scyllo-inositol compound, or a pharmaceutically acceptable salt thereof, or a composition comprising one or more scyllo-inositol compound, and a pharmaceutically acceptable carrier, excipient, or vehicle.

In an embodiment, a method is provided for treating a mammal in need of improved memory, wherein said mammal has no diagnosed disease, disorder, infirmity or ailment known to impair or otherwise diminish memory, comprising the step of administering to the mammal an effective memory-improving amount of one or more scyllo-inositol compound, a pharmaceutically acceptable salt thereof, or a dietary supplement comprising one or more scyllo-inositol compound or a nutraceutically acceptable derivative thereof.

In another aspect of the invention, a method is provided for treating in a subject a condition of the central or peripheral nervous system or systemic organ associated with a disorder in protein folding or aggregation, or amyloid formation, deposition, accumulation, or persistence, comprising administering to the subject a therapeutically effective amount of one or more scyllo-inositol compound, or a pharmaceutically acceptable salt thereof, or a composition comprising one or more scyllo-inositol compound and a pharmaceutically acceptable carrier, excipient, or vehicle.

In a further aspect, the invention provides a method involving administering to a subject a therapeutic compound of one or more scyllo-inositol compound, or pharmaceutically acceptable salts thereof, or a composition comprising one or more scyllo-inositol compound, and a pharmaceutically acceptable carrier, excipient, or vehicle which improves cognitive function, reduces vascular load, reduces astrogliosis, reduces amyloid burden, reduces microgliosis, and/or improves survival.

In a further aspect, the invention provides a method involving administering to a subject a therapeutic compound of one or more scyllo-inositol compound, or pharmaceutically acceptable salts thereof, or a composition comprising one or more scyllo-inositol compound, and a pharmaceutically acceptable carrier, excipient, or vehicle which inhibit amyloid formation, deposition, accumulation and/or persistence, and/or which cause dissolution/disruption of pre-existing amyloid. Thus, the compounds and compositions of the invention may be used for inhibiting amyloidosis in disorders in which amyloid deposition occurs.

In another aspect, the invention provides a method for treating in a subject a condition associated with an amyloid interaction that can be disrupted or dissociated with a compound of the invention comprising administering to the subject a therapeutically effective amount of one or more scyllo-inositol compound, a pharmaceutically acceptable salt thereof, or a composition comprising one or more scyllo-inositol compound and a pharmaceutically acceptable carrier, excipient, or vehicle.

In an aspect, the invention provides a method for preventing, reversing, reducing or inhibiting vascular load, astrogliosis, amyloid burden, and/or microgliosis in a subject comprising administering a therapeutically effective amount of one or more scyllo-inositol compound, a pharmaceutically acceptable salt thereof, or a composition comprising one or more scyllo-inositol compound, and a pharmaceutically acceptable carrier, excipient, or vehicle.

In an aspect, the invention provides a method for increasing or maintaining synaptic function in a subject comprising administering a therapeutically effective amount of one or more scyllo-inositol compound, a pharmaceutically acceptable salt thereof, or a composition comprising one or more scyllo-inositol compound, and a pharmaceutically acceptable carrier, excipient, or vehicle.

The invention has particular applications in treating an amyloid-related disease, in particular Alzheimer's disease. Thus, the invention relates to a method of treatment comprising administering a therapeutically effective amount of one or more scyllo-inositol compound, a pharmaceutically acceptable salt thereof, or a composition comprising a scyllo-inositol compound and a pharmaceutically acceptable carrier, excipient, or vehicle, which upon administration to a subject with symptoms of an amyloid-related disease in particular Alzheimer's disease, produces beneficial effects, preferably sustained beneficial effects. In an embodiment, beneficial effects are evidenced by one or more of the following: improved cognitive function, reduced vascular load, reduced astrogliosis, reduced amyloid burden, reduced microgliosis, and/or improved survival.

In an aspect, the invention provides a method for amelioriating progression of an amyloid-related disease or obtaining a less severe stage of a disease in a subject suffering from such disease (e.g. Alzheimer's disease) comprising administering a therapeutically effective amount of one or more scyllo-inositol compound, a pharmaceutically acceptable salt thereof, or a composition comprising one or more scyllo-inositol compound, and a pharmaceutically acceptable carrier, excipient, or vehicle.

In another aspect, the invention relates to a method of delaying the progression of an amyloid-related disease (e.g. Alzheimer's disease) comprising administering a therapeutically effective amount of one or more scyllo-inositol compound, a pharmaceutically acceptable salt thereof, or a composition comprising one or more scyllo-inositol compound, and a pharmaceutically acceptable carrier, excipient, or vehicle.

In a further aspect, the invention relates to a method of increasing survival of a subject suffering from an amyloid-related disease comprising administering a therapeutically effective amount of one or more scyllo-inositol compound, a pharmaceutically acceptable salt thereof, or a composition comprising one or more scyllo-inositol compound, and a pharmaceutically acceptable carrier, excipient, or vehicle.

In an embodiment, the invention relates to a method of improving the lifespan of a subject suffering from an amyloid-related disease (e.g., Alzheimer's disease) comprising administering a therapeutically effective amount of one or more scyllo-inositol compound, a pharmaceutically acceptable salt thereof, or a composition comprising one or more scyllo-inositol compound, and a pharmaceutically acceptable carrier, excipient, or vehicle.

In an aspect the invention provides a method for treating mild cognitive impairment (MCI) comprising administering a therapeutically effective amount of one or more scyllo-inositol compound, a pharmaceutically acceptable salt thereof, or a composition comprising one or more scyllo-inositol compound, and a pharmaceutically acceptable carrier, excipient, or vehicle.

In an embodiment, the invention provides a method of reducing or reversing amyloid deposition and neuropathology after the onset of cognitive deficits and amyloid plaque neuropathology in a subject comprising administering to the subject a therapeutically effective amount of one or more scyllo-inositol compound, a pharmaceutically acceptable salt thereof, or a composition comprising one or more scyllo-inositol compound and a pharmaceutically acceptable carrier, excipient, or vehicle.

In another embodiment, the invention provides a method of reducing or reversing amyloid deposition and neuropathology after the onset of cognitive deficits and amyloid plaque neuropathology in a subject comprising administering to the subject an amount of one or more scyllo-inositol compound, a pharmaceutically acceptable salt thereof, or a composition comprising one or more scyllo-inositol compound and a pharmaceutically acceptable carrier, excipient, or vehicle effective to reduce or reverse amyloid deposition and neuropathology after the onset of cognitive deficits and amyloid plaque neuropathology.

Aspects of the invention provide improved methods and compositions for use of one or more scyllo-inositol compound for sustained treatment of a disorder and/or disease (e.g., Alzheimer's disease). The present invention in an embodiment provides a composition comprising one or more scyllo-inositol compound that achieves greater efficacy, potency, and utility. For example, the greater efficacy can be shown by improving or reversing cognitive decline and/or survival in Alzheimer's disease with treatment resulting in sustained improvement and/or increased survival after ceasing treatment.

In an aspect of the invention a compound of the formula Ia or Ib is utilized in the treatment of Alzheimer's disease. Thus, Alzheimer's disease may be treated by administering a therapeutically effective amount of a compound of the formula Ia or formula Ib. Such treatment may be effective for retarding the degenerative effects of Alzheimer's disease, including specifically, but not exclusively, deterioration of the central nervous system, loss of mental facilities, loss of short term memory, and disorientation.

In an embodiment, where the disease is Alzheimer's disease, dementia or MCI, beneficial effects of a compound or composition or treatment of the invention can manifest as at least one, two, three, four, five, six, seven, eight, nine, ten, twelve, thirteen, fourteen, fifteen, or all of the following, in particular five or ten or more, more particularly fifteen or more of the following:
a) An increase or restoration of long term potentiation relative to the level in the absence of a compound disclosed herein after administration to a subject with symptoms of Alzheimer's disease. In aspects of the invention the compounds induce at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in long term potentiation in a subject.
b) An increase or maintenance of synaptic function relative to the level of synaptic function in the absence of a compound disclosed herein after administration to a subject with symptoms of Alzheimer's disease. In aspects of the invention the compounds induce at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, 99%, 100%, 125%, 150%, 175% or 200% increase in synaptic function in a subject.
c) An increase in synaptophysin. In aspects of the invention there is at least about a 2%, 5%, 10%, 15%, 20%, 30%, 40%, 50%, 60%, 70%, 80%, 90%, 95%, 99%, 100%, 125%, 150%, 175% or 200% increase in synaptophysin.
d) An increase in synaptophysin reactive boutons and cell bodies. In aspects of the invention there is at least about a 2%, 5%, 10%, 15%, 20%, 30%, 40%, 50%, 60%, 70%, 80%, 90%, 95%, 99%, 100%, 125%, 150%, 175% or 200%, more particularly about a 100-150% or 140-150%, increase in synaptophysin reactive boutons and cell bodies.
e) A reduction, slowing or prevention of an increase in, or an absence of symptoms of inflammation, in particular an Aβ-induced inflammatory response, after administration to a subject with symptoms of Alzheimer's disease.
f) A reduction, slowing or prevention of an increase in cerebral accumulation of amyloid β relative to the levels measured in the absence of a compound disclosed herein in subjects with symptoms of Alzheimer's disease. In aspects of the invention, the compound induces at least about a 2%, 5%, 10%, 15%, 20%, 30%, 40%, 50%, 60%, 70%, 80%, or 90% decrease in cerebral accumulation of amyloid P.
g) A reduction, slowing or prevention of an increase in deposition of cerebral amyloid plaques, relative to the levels measured in the absence of a compound disclosed herein in subjects with symptoms of Alzheimer's disease. In aspects of the invention, the compound induces at least about a 2%, 5%, 10%, 15%, 20%, 30%, 40%, 50%, 60%, 70%, 80%, or 90% decrease in deposition of cerebral amyloid plaques.
h) A reduction, slowing or prevention of an increase in plaque number. In aspects of the invention, a compound disclosed herein induces at least about a 2%, 5%, 10%, 15%, 20%, 30%, 40%, 50%, 60%, 70%, 80%, or 90% reduction in plaque number. In particular aspects the compound induces a 5-15% or 10-15% reduction in plaque number.
i) A reduction, slowing or prevention of an increase in plaque size. In aspects of the invention, a compound disclosed herein induces at least about a 2%, 5%, 10%, 15%, 20%, 30%, 40%, 50%, 60%, 70%, 80%, or 90% reduction in plaque size. In particular aspects the compound induces a 5-15% or 10-15% reduction in plaque size.
j) A reduction, slowing or prevention of an increase in percent area of the brain covered in plaques. In aspects of the invention, a compound disclosed herein induces at least about a 2%, 5%, 10%, 15%, 20%, 30%, 40%, 50%, 60%, 70%, 80%, or 90% reduction in percent area of the brain covered in plaques. In particular aspects the compound induces a 5-15% or 10-15% reduction in percent area of the brain covered in plaques.
k) A reduction, slowing or prevention of an increase in soluble Aβ oligomers in the brain, relative to the levels measured in the absence of a compound disclosed herein in subjects with symptoms of Alzheimer's disease. In aspects of the invention, the combination induces at least about a 2%, 5%, 10%, 15%, 20%, 30%, 40%, 50%, 60%, 70%, 80%, or 90% decrease in soluble Aβ oligomers.
l) A reduction, slowing or prevention of an increase in brain levels of Aβ40. In aspects of the invention, a compound disclosed herein induces at least about a 2%, 5%, 10%, 15%, 20%, 30%, 40%, 50%, 60%, 70%, 80%, or 90% reduction in Aβ40. In particular aspects the compound induces a 10-50%, 20-45%, or 25-35% reduction in brain levels of Aβ40.
m) A reduction, slowing or prevention of an increase in Aβ42 levels in a body fluid such as CSF or blood. In aspects of the invention, a compound disclosed herein induces at least about a 2%, 5%, 10%, 15%, 20%, 30%, 40%, 50%, 60%, 70%, 80%, or 90% reduction in Aβ42. In particular aspects the compound induces a 10-50%, 15-40%, or 20-25% reduction in brain levels of Aβ42.
n) A reduction, slowing or prevention of an increase in brain levels of Aβ42. In aspects of the invention, a compound disclosed herein induces at least about a 2%, 5%, 10%, 15%, 20%, 30%, 40%, 50%, 60%, 70%, 80%, or 90% reduction in Aβ42. In particular aspects the compound induces a 10-50%, 15-40%, or 20-25% reduction in brain levels of Aβ42.
o) A reduction, slowing or prevention of an increase in glial activity in the brain, relative to the levels measured in the absence of a compound disclosed herein in subjects with symptoms of Alzheimer's disease. Preferably, the compound induces at least about a 2%, 5%, 10%, 15%, 20%, 30%, 40%, 50%, 60%, 70%, 80%, or 90% decrease in glial activity
p) Maintenance of synaptic function at about normal for a prolonged period of time, in particular for at least 5 weeks, 6 weeks, 8 weeks, 10 weeks, 12 weeks, 14 weeks, 16 weeks, 20 weeks, 24 weeks, 30 weeks, 40 weeks, 52 weeks, or 78 weeks, more particularly, 2 to 4 weeks, 2 to 5 weeks, 3 to 5 weeks, 2 to 6 weeks, 2 to 8 weeks, 2 to 10 weeks, 2 to 12 weeks, 2 to 16 weeks, 2 to 20 weeks, 2 to 24 weeks, 2 weeks to 12 months, or 2 weeks to 24 months following treatment.
q) A reduction or slowing of the rate of disease progression in a subject with Alzheimer's disease. In particular a reduction or slowing of cognitive decline in a subject with Alzheimer's disease.
r) A reduction in totat vascular load. In aspects of the invention, the compounds induce at least about a 2%, 5%, 10%, 15%, 20%, 30%, 40%, 50%, 60%, 70%, 80%, or 90% reduction in total vascular load.
s) A reduction in astrogliosis. In aspects of the invention, the compounds induce at least about a 2%, 5%, 10%, 15%, 20%, 30%, 40%, 50%, 60%, 70%, 80%, or 90% reduction in astrogliosis.
t) A reduction in microgliosis. In aspects of the invention, the compounds induce at least about a 2%, 5%, 10%, 15%, 20%, 30%, 40%, 50%, 60%, 70%, 80%, or 90% reduction in microgliosis.
u) A reduction or slowing of cognitive deficits.
v) A reduction in or slowing of amyloid angiopathy.
w) A reduction in accelerated mortality.
x) An increase in survival in a subject with symptoms of Alzheimer's disease. Preferably, the compounds induce at least about a 20%, 30%, 40%, 50%, 60%, 70%, 75%, 80%, 90%, or 95% increase in survival.

In aspects of the invention beneficial effects of a composition or treatment of the invention can manifest as (a) and (b); (a), (b) and (c); (a), (b), (e), (f) and (g); (a), (b), (e), (f) through (h); (a), (b), (e), (f) through (i); (a), (b), (e), (f) through (j); (a), (b), (e), (f) through (k); (a), (b), (e), (f) through (l); (a), (b), (e), (f) through (m); (a), (b), (e), (f) through (n); (a), (b), (e), (f) through (o); (a), (b), (e), (f) through (p); (a), (b), (e), (f) through (q); (a), (b), (e), (f) through (r); (a), (b), (e), (f) through (s); (a), (b), (e), (f) through (t); (a) through (d); (a) through (e); (a) through (f); (a) through (g); (a) through (h); (a) through (i); (a) through (j); (a) through (k); (a) through (l); (a) through (m); (a) through (n); (a) through (o); (a) through (p); (a) through (q); (a) through (r); (a) through (s); (a) through (t); (a) through (u); (a) through (v); (a) through (w) or (a) through (x).

Compounds, pharmaceutical compositions and methods of the invention can be selected that have statistically significant beneficial effects, in particular one or more of the effects of (a) through (x) above. Compounds, pharmaceutical compositions and methods of the invention can also be selected that have sustained beneficial effects, in particular statistically significant sustained beneficial effects. In an embodiment, a pharmaceutical composition is provided with statistically significant sustained beneficial effects, in particular sustained beneficial effects of one or more of (a) through (x) above, comprising a therapeutically effective amount of one or more scyllo-inositol compound. In aspects of the invention, one or more of the beneficial effects provide enhanced therapeutic effects compared with conventional therapies.

Greater efficacy and potency of a treatment of the invention in some aspects may improve the therapeutic ratio of treatment, reducing untoward side effects and toxicity. Selected methods of the invention may also improve long-standing Alzheimer's disease even when treatment is begun long after the appearance of symptoms. Prolonged efficacious treatment can be achieved in accordance with the invention following administration of a compound or composition of the invention.

In a further aspect, the invention provides a method for treating Alzheimer's disease in a patient in need thereof which includes administering to the individual a composition that provides one or more scyllo-inositol compound in a dose sufficient to improve cognitive function, reduce vascular load, reduce astrogliosis, reduce amyloid burden, reduce microgliosis, and/or improve survival. In another aspect, the invention provides a method for treating Alzheimer's disease comprising administering, preferably orally or systemically, an amount of a scyllo-inositol compound to a mammal, to improve cognitive function, reduce vascular load, reduce astrogliosis, reduce amyloid burden, reduce microgliosis, and/or improve survival for a prolonged period following administration.

In an aspect, the invention relates to a method for treating Alzheimer's disease comprising contacting Aβ, Aβ aggregates, or Aβ oligomers in particular Aβ40 or Aβ40 aggregates or oligomers and/or Aβ42 or Aβ42 aggregates or oligomers, in a subject with a therapeutically effective amount of one or more scyllo-inositol compound or a composition comprising an epi-inositol compound.

In another aspect, the invention provides a method for treating Alzheimer's disease by providing a composition comprising one or more one or more scyllo-inositol compound in an amount sufficient to disrupt aggregated AD or Aβ oligomers for a prolonged period following administration.

In a further aspect, the invention provides a method for treating Alzheimer's disease in a patient in need thereof which includes administering to the individual a composition that provides one or more one or more scyllo-inositol compound in a dose sufficient to increase or restore long term potentiation and/or maintain synaptic function. In another aspect, the invention provides a method for treating Alzheimer's disease comprising administering, preferably orally or systemically, an amount of one or more scyllo-inositol compound to a mammal, to reduce cerebral accumulation of Aβ, deposition of cerebral amyloid plaques, soluble Aβ oligomers in the brain, glial activity, and/or inflammation for a prolonged period following administration.

The invention in an embodiment provides a method for treating Alzheimer's disease, the method comprising administering to a mammal in need thereof a composition comprising one or more scyllo-inositol compound in an amount sufficient to reduce cognitive decline, especially for a prolonged period following administration, thereby treating the Alzheimer's disease.

The invention in an embodiment provides a method for treating Alzheimer's disease, the method comprising administering to a mammal in need thereof a composition comprising one or more one or more scyllo-inositol compound in an amount sufficient to increase or maintain synaptic function, especially for a prolonged period following administration, thereby treating the Alzheimer's disease.

In another aspect, the invention provides a method for preventing and/or treating Alzheimer's disease, the method comprising administering to a mammal in need thereof a composition comprising one or more one or more scyllo-inositol compound in an amount sufficient to disrupt aggregated Aβ or Aβ oligomers for a prolonged period following administration; and determining the amount of aggregated Aβ or Aβ oligomers, thereby treating the Alzheimer's disease. The amount of aggregated Aβ or Aβ oligomers may be measured using an antibody specific for Aβ or an epi-inositol labeled with a detectable substance.

The present invention also includes methods of using the compositions of the invention in combination treatments with one or more additional therapeutic agents including without limitation beta-secretase inhibitors, gamma-secretase inhibitors, epsilon-secretase inhibitors, other inhibitors of beta-sheet aggregation/fibrillogenesis/ADDL formation (e.g. Alzhemed), NMDA antagonists (e.g. memantine), non-steroidal anti-inflammatory compounds (e.g. Ibuprofen, Celebrex), anti-oxidants (e.g. Vitamin E), hormones (e.g. estrogens), nutrients and food supplements (e.g. Gingko biloba), statins and other cholesterol lowering drugs (e.g. Lovastatin and Simvastatin), acetylcholinesterase inhibitors (e.g. donezepil), muscarinic agonists (e.g. AF102B (Cevimeline, EVOXAC), AF150(S), and AF267B), anti-psychotics (e.g. haloperidol, clozapine, olanzapine), anti-depressants including tricyclics and serotonin reuptake inhibitors (e.g. Sertraline and Citalopram Hbr), statins and other cholesterol lowering drugs (e.g. Lovastatin and Simvastatin), immunotherapeutics and antibodies to Aβ (e.g. ELAN AN-1792), vaccines, inhibitors of kinases (CDK5, GSK3a, GSK30) that phosphorylate TAU protein (e.g. Lithium chloride), inhibitors of kinases that modulate Aβ production (GSK3α, GSK3β, Rho/ROCK kinases) (e.g. lithium Chloride and Ibuprofen), drugs that upregulate neprilysin (an enzyme which degrades Aβ); drugs that upregulate insulin degrading enzyme (an enzyme which degrades Aβ), agents that are used for the treatment of complications resulting from or associated with a disease, or general medications that treat or prevent side effects. The present invention also includes methods of using the compositions of the invention in combination treatments with one or more additional treatments including without limitation gene therapy and/or drug based approaches to upregulate neprilysin (an enzyme which degrades Aβ), gene therapy and/or drug based approaches to upregulate insulin degrading enzyme (an enzyme which degrades Aβ), or stem cell and other cell-based therapies.

Combinations of a scyllo-inositol compound and a therapeutic agent or treatment may be selected to provide unexpectedly additive effects or greater than additive effects i.e. synergistic effects. Other therapeutics and therapies may act via a different mechanism and may have additive/synergistic effects with the present invention

A composition or method (i.e., combination treatment) comprising one or more scyllo-inositol compound and a therapeutic agent employing different mechanisms to achieve maximum therapeutic efficacy, may improve tolerance to the therapy with a reduced risk of side effects that may result from higher doses or longer term monotherapies (i.e. therapies with each compound alone). A combination treatment may also permit the use of lower doses of each compound with reduced adverse toxic effects of each compound. A suboptimal dosage may provide an increased margin of safety, and may also reduce the cost of a drug necessary to achieve prophylaxis and therapy. In addition, a treatment utilizing a single combination dosage unit may provide increased convenience and may result in enhanced compliance. Other advantages of a combination therapy may include higher stability towards degradation and metabolism, longer duration of action, and/or longer duration of action or effectiveness at particularly low doses.

In an aspect, the invention contemplates the use of a composition comprising at least one scyllo-inositol compound for the preparation of a medicament in treating a disorder and/or disease. The invention also contemplates the use of a composition comprising at least one scyllo-inositol compound for the preparation of a medicament for preventing and/or treating disorders and/or diseases. The invention additionally provides uses of a pharmaceutical composition of the invention in the preparation of medicaments for the prevention and/or treatment of disorders and/or diseases. The medicaments provide beneficial effects, preferably sustained beneficial effects following treatment. The medicament may be in a form for consumption by a subject such as a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder for inhibition of amyloid formation, deposition, accumulation, and/or persistence, regardless of its clinical setting.

In an embodiment, the invention relates to the use of a therapeutically effective amount of at least one scyllo-inositol compound or a composition of the invention for preparation of a medicament for providing therapeutic effects, in particular beneficial effects, preferably sustained beneficial effects, in an amyloid-related disease.

In another embodiment the invention provides the use of one or more scyllo-inositol compound or composition of the invention for the preparation of a medicament for prolonged or sustained treatment of Alzheimer's disease.

In a further embodiment the invention provides the use of a scyllo-inositol compound for preparation of a pharmaceutical composition to be employed through oral administration for treatment of a disorder characterized by abnormal protein folding and/or aggregation, and/or amyloid formation, deposition, accumulation, or persistence.

Therapeutic efficacy and toxicity of compositions and methods of the invention may be determined by standard pharmaceutical procedures in cell cultures or with experimental animals such as by calculating a statistical parameter such as the ED50 (the dose that is therapeutically effective in 50% of the population) or LD50 (the dose lethal to 50% of the population) statistics. The therapeutic index is the dose ratio of therapeutic to toxic effects and it can be expressed as the ED50/LD50 ratio. Pharmaceutical compositions which exhibit large therapeutic indices are preferred. One or more of the therapeutic effects, in particular beneficial effects disclosed herein, can be demonstrated in a subject or disease model. For example, beneficial effects may be demonstrated in a model described in the Examples herein, in particular beneficial effects may be demonstrated in a TgCRND8 mouse with symptoms of Alzheimer's disease.

### Administration

Scyllo-inositol compounds and compositions of the present invention can be administered by any means that produce contact of the active agent(s) with the agent's sites of action in the body of a subject or patient to produce a therapeutic effect, in particular a beneficial effect, in particular a sustained beneficial effect. The active ingredients can be administered simultaneously or sequentially and in any order at different points in time to provide the desired beneficial effects. A compound and composition of the invention can be formulated for sustained release, for delivery locally or systemically. It lies within the capability of a skilled physician or veterinarian to select a form and route of administration that optimizes the effects of the compositions and treatments of the present invention to provide therapeutic effects, in particular beneficial effects, more particularly sustained beneficial effects.

The compounds and compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. The compositions of the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen. A sustained release formulation can also be used for the therapeutic agents.

In aspects of the invention the compounds and compositions are administered by peripheral administration, in particular by intravenous administration, intraperitoneal administration, subcutaneous administration, intramuscular administration, oral administration, topical administration, transmucosal administration, or pulmonary administration.

The dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect.

An amount of a scyllo-inositol compound or composition comprising same which will be effective in the treatment of a particular disorder and/or disease to provide effects, in particular beneficial effects, more particularly sustained beneficial effects, will depend on the nature of the disorder and/or disease, and can be determined by standard clinical techniques. The precise dose to be employed in the formulation will also depend on the route of administration, and the seriousness of the disease, and should be decided according to the judgment of the practitioner and each patient's circumstances.

Suitable dosage ranges for administration are particularly selected to provide therapeutic effects, in particular beneficial effects, more particularly sustained beneficial effects. A dosage range is generally effective for triggering the desired biological responses. The dosage ranges are generally about 0.5 mg to about 2 g per kg, about 1 mg to about 1 g per kg, about 1 mg to about 200 mg per kg, about 1 mg to about 100 mg per kg, about 1 mg to about 50 mg per kg, about 10 mg to about 100 mg per kg, or about 30 mg to 70 mg per kg of the weight of a subject administered once, twice, three times or more daily.

In some aspects of the invention, the dosage ranges of a compound disclosed herein administered once twice, three times or more daily, especially once or twice daily, are about 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 2 to 35 mg/kg, 2.5 to 30 mg/kg, 3 to 30 mg/kg, 3 to 20 mg/kg, or 3 to 15 mg/kg.

In embodiments of the invention, the required dose of a compound disclosed herein administered twice daily is about 1 to 50 mg/kg, 1 to 40 mg/kg, 2.5 to 40 mg/kg, 3 to 40 mg/kg, 3 to 35 mg/kg, most preferably 3 to 30 mg/kg. In embodiments of the invention, the required daily dose of the compound is about 1 to 80 mg/kg and within that range 1 to 70 mg/kg, 1 to 65 mg/kg, 2 to 70 mg/kg, 3 to 70 mg/kg, 4 to 65 mg/kg, 5 to 65 mg/kg, or 6 to 60 mg/kg.

In embodiments of the invention, the required dose of a compound disclosed herein, administered twice daily is about 1 to 50 mg/kg, 1 to 40 mg/kg, 2.5 to 40 mg/kg, 3 to 40 mg/kg, 3 to 35 mg/kg, most preferably 3 to 30 mg/kg.

In other embodiments of the invention, the required daily dose of a compound disclosed herein, is about 1 to 80 mg/kg and within that range 1 to 70 mg/kg, 1 to 65 mg/kg, 2 to 70 mg/kg, 3 to 70 mg/kg, 4 to 65 mg/kg, 5 to 65 mg/kg, or 6 to 60 mg/kg.

A composition or treatment of the invention may comprise a unit dosage of at least one scyllo-inositol compound to provide beneficial effects, in particular one or more of the beneficial effects (a) to (t) set out herein. A “unit dosage” or “dosage unit” refers to a unitary i.e., a single dose which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A subject may be treated with a scyllo-inositol compound or composition or formulation thereof on substantially any desired schedule. A composition of the invention may be administered one or more times per day, in particular 1 or 2 times per day, once per week, once a month or continuously. However, a subject may be treated less frequently, such as every other day or once a week, or more frequently.

A scyllo-inositol compound, composition or formulation of the invention may be administered to a subject for about or at least about 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, or 2 weeks to 24 months, periodically or continuously.

In an aspect, the invention provides a regimen for supplementing a human's diet, comprising administering to the human a supplement comprising a scyllo-inositol compound, or nutraceutically acceptable derivatives thereof. A subject may be treated with a supplement at least about every day, or less frequently, such as every other day or once a week. A supplement of the invention may be taken daily but consumption at lower frequency, such as several times per week or even isolated doses, may be beneficial.

In a particular aspect, the invention provides a regimen for supplementing a human's diet, comprising administering to the human about 25 to about 200 milligrams of a compound of the formula Ia or Ib, or nutraceutically acceptable derivatives thereof on a daily basis. In another aspect, about 50-100 milligrams of a compound of the formula Ia or Ib is administered to the human on a daily basis.

A supplement of the present invention may be ingested with or after a meal. Thus, a supplement may be taken at the time of a person's morning meal, and/or at the time of a person's noontime meal. A portion may be administered shortly before, during, or shortly after the meal. For daily consumption, a portion of the supplement may be consumed shortly before, during, or shortly after the human's morning meal, and a second portion of the supplement may be consumed shortly before, during, or shortly after the human's noontime meal. The morning portion and the noontime portion can each provide approximately the same quantity of a scyllo-inositol compound. A supplement and regimens described herein may be most effective when combined with a balanced diet according to generally accepted nutritional guidelines, and a program of modest to moderate exercise several times a week.

In an embodiment, a regimen for supplementing a human's diet is provided comprising administering to the human a supplement comprising, per gram of supplement: about 5 milligram to about 30 milligrams of one or more scyllo-inositol compound or a nutraceutically acceptable derivative thereof. In an embodiment, a portion of the supplement is administered at the time of the human's morning meal, and a second portion of the supplement is administered at the time of the human's noontime meal.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLE 1

The following methods were used in the studies described in this example:

Mice. Experimental groups of TgCRND8 mice [12, 13] on a C3H/B6 outbred background were initially treated with either epi- or scyllo-cyclohexanehexol 30 mg/day. This initial dosage was chosen based upon the dosage of myo-cyclohexanehexol (6-18 grams/day/adult or 86-257 mg/Kg/day) that is typically administered to human patients for various psychiatric disorders [35]. In these dosages, myo-cyclohexanehexol had no toxicity in humans or animals. The studies described herein were repeated using doses of 5 mg/Kg/day-100 mg/Kg/day, and these alternate doses have generated the same results (data not shown). A cohort of animals (n=10 mice per treatment arm) entered the study at five months of age, and outcomes were then analyzed after one month of treatment. The body weight, coat characteristics and in cage behaviour were monitored. Mannitol was used as a negative control for potential alterations in caloric intake. All experiments were performed according to the Canadian Council on Animal Care guidelines.

Behavioural tests: Morris Water Maze testing was performed as previously described [14]. After non-spatial pre-training, mice underwent place discrimination training for 5 days with 4-trials per day, followed by a cued visible platform to rule out general motivational, learning deficits and motor problems, and a probe trial to evaluate memory. Data were subjected to a mixed model of repeated measures analysis of variance (ANOVA) with treatment (untreated, epi- or scyllo-cyclohexanehexol) and genotype (TgCRND8 versus non-Tg) as ‘between-subject’ factors. Open field test for motor activity was preformed as described previously [28]. Duration of walking, pausing and grooming were analyzed as indices of spontaneous locomotor activity. Sensorimotor function was examined with an Economex™ accelerating rotarod (Columbus Instruments, Columbus, Ohio), as described elsewhere [29]. The rod was set to accelerate at a rate of 0.2 r.p.m./s, from an initial, constant speed of 5 r.p.m. Latency to fall was recorded in four daily trials, conducted at 30 min intervals. All mice were trained for seven days before testing. The test day performance score for each animal was obtained by summing its latency to fall over the four trials

Cerebral amyloid burden. Brains were removed and one hemisphere was fixed in 4% paraformaldehyde and embedded in paraffin wax in the mid sagittal plane. To generate sets of systematic uniform random sections, 5 μm serial sections were collected across the entire hemisphere. Sets of sections at 50 μm intervals were used for analyses (10-14 sections/set). Plaques were identified after antigen retrieval with formic acid, and incubation with primary anti-Aβ antibody (Dako M-0872), followed by secondary antibody (Dako StreptABCcomplex/horseradish kit). End products were visualized with DAB and were counter-stained with luxol fast blue. Amyloid plaque burden was assessed with Leco IA-3001 image analysis software interfaced with Leica microscope and Hitachi KP-M1U CCD video camera. Openlab imaging software (Improvision, Lexington, Mass.) was then used to convert micrographs to binary images for plaque number and plaque area determinations. Vascular amyloid burden was defined as amyloid originating from or surrounding blood vessels and was analysed similarly.

Plasma and Cerebral Aβ Content. Hemi-brain samples were homogenized in a buffered sucrose solution, followed by either 0.4% diethylamine/100 mM NaCl for soluble Aβ levels or cold formic acid for the isolation of total Aβ. After neutralization, the samples were diluted and analyzed for Aβ40 and Aβ42 using commercially available kits (BIOSOURCE International). Each hemisphere was analyzed in triplicate and the mean values±SEM reported. Western blot analyses were performed on all fractions using urea gels for Aβ species analyses [30]. Aβ was detected using 6E10 (BIOSOURCE International) and Enhanced Chemiluminenscence (Amersham).

Gliosis Quantitation. Five randomly selected, evenly spaced, sagittal sections were collected from paraformaldehyde-fixed and frozen hemispheres of treated and control mice. Sections were immunolabelled for astrocytes with anti-rat GFAβ IgG2a (Dako; diluted 1:50) and for microglia with anti-rat CD68 IgG2b (Dako; 1:50). Digital images were captured using a Coolsnap digital camera (Photometrics, Tuscon, Ariz.) mounted to a Zeiss, Axioscope 2 Plus microscope. Images were analysed using Openlab 3.08 imaging software (Improvision, Lexington Mass.).

Survival Census: The probability of survival was assessed by the Kaplan-Meier technique [31], computing the probability of survival at every occurrence of death, thus making it suitable for small sample sizes. For the analyses of survival, 35 mice were used for each treatment group. The Tarone-Ware test was used to assess effects of treatments.

Analysis of APP in brain. Mouse hemi-brain samples were homogenized and spun at 109,000×g, in 20 mM Tris pH7.4,0.25M sucrose, 1 mM EDTA and 1 mM EGTA, and a protease inhibitor cocktail, mixed with 0.4% DEA (diethylamine)/100 mM NaCl. The supernatants were analysed for APPs levels by Western blotting using mAb 22C11, while the pellets were analysed for APP holoprotein with mAb C1/6.1 as previously described [12,13].

Soluble Aβ oligomer Analyses. The levels of soluble Aβ oligomers were measured by a dot blot assay with anti-oligomer specific antibodies [16]. Briefly, oligomers were solubilised from one hemi-brain in PBS in the presence of protease inhibitor cocktail (Sigma). After centrifugation at 78,500×g for 1 hr at 4° C., the supernatants were analysed. Protein content was determined by the BCA protein assay (Pierce). Two μg of total protein was spotted onto nitrocellulose, blocked with 10% non-fat milk in TBS before incubation with the biotinylated oligomeric specific antibody. Blots were incubated with streptavidin-HRP and ECL chemiluminescence kit. Soluble and fibrillar Aβ42 were used as negative controls and synthetic oligomeric Aβ42 was used as a positive control [20]. Control samples were re-identified after oligomeric antibody was stripped and re-probing with the anti-Aβ antibody 6E10.

Long Term Potentiation. Field potentials were recorded in CA1 of mouse hippocampus by standard procedures [39,40]. Swiss Webster mice between the ages of P16 and P26 were anesthetized with isoflurane. The brain was rapidly removed and placed in ice cold oxygenated sucrose-CSF containing (in mM): 248 sucrose, 2 KCl, 2 MgSO4, 1.24 NaH2PO4, 1 CaCl₂, 1 MgCl2, 26 NaHCO3, 10 D-glucose, pH 7.4, ˜315 mOsmol [41]. The hippocampus from each hemisphere was isolated and 350 μm coronal sections were made. The slices were transferred to a holding chamber containing NaCl—CSF (in mM: 124 NaCl, 2 KCl, 2 MgSO4, 1.25 NaH2PO4, 2 CaCl2, 26 NaHCO3, 10 D-glucose, pH 7.4, ˜310 mOsmol) and allowed to recover for more than 1 hour. Once placed in the chamber, slices were continuously perfused by a closed loop containing 15 ml of ACSF to conserve the oligomeric Aβ. After 20 minutes of stable baseline, 1 ml of 15× concentrated 7PA2 conditioned medium±1.25 μM scyllo-cyclohexanehexol was added to the perfusion loop. A bipolar stimulating electrode (World Precision Inst.) was placed in the Schaffer collaterals to deliver baseline stimuli and tetani. A borosilicate glass recording electrode (2-4 MΩ) containing ACSF was positioned approximately 75-200 μm from the stimulating electrode. The intensity of the stimulus (typically between 10-20 μAmps) was set to obtain 25-40% of the maximal field potential response. Test stimuli were delivered at 0.05 Hz. To induce LTP, 4 tetani (100 Hz for 1 second) were delivered 5 minutes apart. Field potential responses were amplified 10× using an Axopatch 200B. The data was sampled at 10 kHz and filtered at 2 kHz. Traces were analyzed using pClamp 9.2. The slope of the field potential was estimated using approximately 10-60% of the total response.

### Synaptophysin Quantification.

Synaptophysin immunohistochemical staining was performed on 3 evenly spaced saggital sections of paraformaldehyde-fixed treated and control mice. Sections were immunolabelled for synaptophysin with anti-synaptophysin IgG (1:40; Roche, Laval, PQ). Digital images were captured and analyzed as described above. Within each section, three randomly chosen 100 μm2 areas of the CA1 region of the hippocampus were counted for synaptophysin reactive cell bodies and boutons. The results are expressed as the mean of the number of reactive bodies and boutons per 100 μm2 [33, 34].

### Results

To assess their effectiveness in vivo, inositol compounds were administered to a robust murine model of Alzheimer's disease (TgCRND8) [12, 13]. TgCRND8 mice express a human amyloid precursor protein transgene (APP695) bearing two missense mutations that cause AD in humans (KM670/671NL and V717F). At about three months of age, the mice display progressive spatial learning deficits that are accompanied both by rising cerebral Aβ levels and by increasing numbers of cerebral extracellular amyloid plaques [12]. By six months of age, the levels of Aβ and the morphology, density and distribution of the amyloid plaques in the brain of TgCRND8 mice are similar to those seen in the brains of humans with well-established AD [13]. As in human patients with AD, the biochemical, behavioural and neuropathological features of the mouse model are accompanied by accelerated mortality [12, 13].

The TgCRND8 mice and non-transgenic littermates were assigned to sex- and age-matched cohorts that were then used to test the effectiveness of the cyclohexanehexol stereoisomers as a therapeutic (with treatment delayed until five months of age and continued for one month until six months of age). The mice were randomly assigned to receive active compound (1,2,3,4,5/6-(epi-) cyclohexanehexol or 1,3,5/2,4,6-(scyllo-) cyclohexanehexol administered orally), mock therapy (mannitol), or no therapy. The endpoints were cognitive function, brain Aβ levels, and neuropathology. 1,2,3,5/4,6-(myo-) cyclohexanehexol was not included in these studies because prior in vitro studies [11] had indicated that myo-cyclohexanehexol was only weakly effective, and because pilot in vivo studies showed no significant benefit (data not shown). Over the course of these experiments, observers were unaware of genotype or treatment group.

### Cyclohexanehexol Stereoisomers Reverse Established Cerebral Amyloid Deposition

Most AD patients will seek treatment only after they have become symptomatic, i.e., at a time when Aβ oligomerization, deposition, toxicity and plaque formation are already well advanced. To assess whether cyclohexanehexol stereoisomers could abrogate a well-established AD-like phenotype, the start of treatment of the TgCRND8 mice was delayed until five months of age. At this age, TgCRND8 mice have significant behavioural deficits, accompanied by profuse Aβ peptide and plaque burdens [12]. Cohorts of TgCRND8 and non-Tg littermates (10 mice per cohort) were either treated for 28 days with epi-cyclohexanehexol or with scyllo-cyclohexanehexol, or were left untreated. The dosage and oral administration of compounds, and the neurochemical and neuropathological assays used for these experiments were the same as those employed in the initial prophylactic experiments. Mortality curves were not generated for this cohort of animals because the brevity of the trial resulted in too few deaths in the untreated TgCRND8 mice to generate meaningful data.

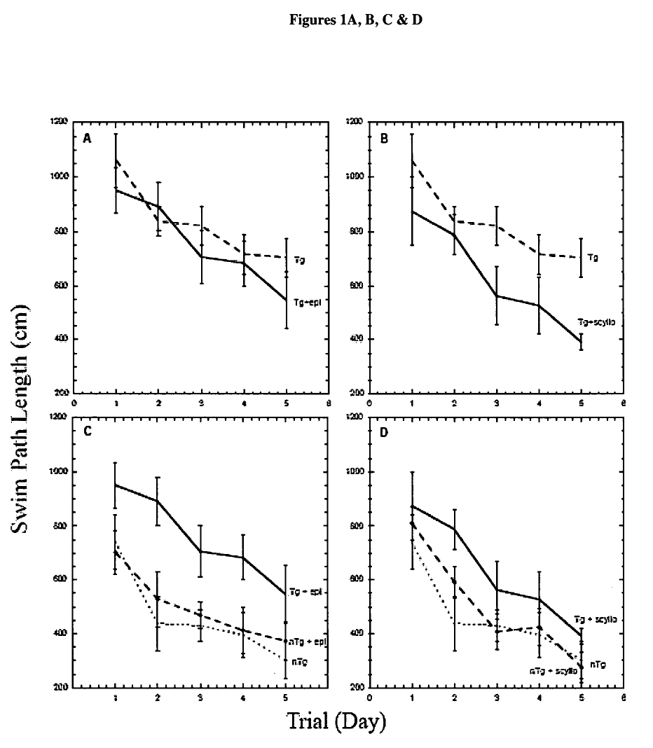

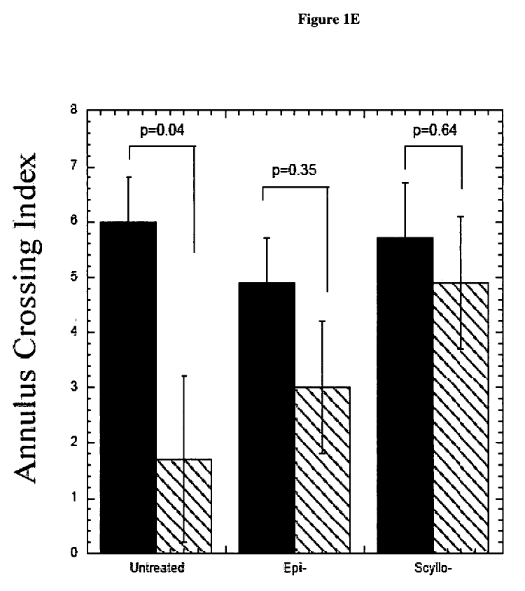

Spatial learning in these mice was compared between six month old TgCRND8 mice that had been treated with epi-cyclohexanehexol or with scyllo-cyclohexanehexol or that were untreated for 28 days. The performance of six month old TgCRND8 mice that had been treated with epi-cyclohexanehexol for 28 days was not significantly different from that of untreated TgCRND8 littermates (F1,15=3.02; p=0.27; FIG. 1A), and was significantly poorer than the performance of their non-Tg littermates (F1,14=11.7, p=0.004; FIG. 1C). Furthermore, the probe trial confirmed that epi-cyclohexanehexol treated TgCRND8 mice were not statistically different from untreated TgCRND8 mice (p=0.52; FIG. 1E). Epi-cyclohexanehexol had no significant impact on brain Aβ40 or Aβ42 levels, percent area of the brain covered with plaques, or plaque number in animals with pre-existing disease (Table 1).

The 28-day treatment of five month old TgCRND8 mice with scyllo-cyclohexanehexol resulted in significantly better behavioural performance compared to untreated TgCRND8 mice (p=0.01). Indeed, the cognitive performance of these scyllo-cyclohexanehexol-treated TgCRND8 mice was indistinguishable from that of their non-Tg littermates (F1,13=2.9, p=0.11; FIGS. 1B, D). This beneficial effect of cyclohexanehexol treatment was not due to non-specific effects on behavioural, motor, or perceptual systems because cyclohexanehexol treatment had no effect on the cognitive performance of non-Tg mice (F2,19=0.98; p=0.39). In the probe trial, the annulus-crossing index showed a significant improvement in memory for scyllo-cyclohexanehexol treated TgCRND8 mice that was not statistically different from non-Tg littermates (p=0.64; FIG. 1E). In a separate cohort of mice and using % time in target quadrant as an alternate measure, scyllo-cyclohexanehexol treated TgCRND8 mice were not statistically different from non-Tg littermates (p=0.28; data not shown). The beneficial effects of scyllo-cyclohexanehexol were not due to alteration of sensorimotor behaviour. Scyllo-cyclohexanehexol had no effect on grooming or activity of TgCRND8 mice in comparison to both untreated TgCRND8 mice (F1,9=0.25; p=0.63) and non-Tg littermates (F1,12=0.02; p=0.89) in the open field test (supplemental data). Similarly, Rotarod testing revealed no difference between scyllo-cyclohexanehexol treated and untreated TgCRND8 mice (p=0.42) or between treated TgCRND8 and treated or untreated non-Tg littermates (p=0.79) in sensorimotor function. In agreement with the results of the prophylactic study, a 28 day course of scyllo-cyclohexanehexol at 5 months of age also: 1) reduced brain levels of Aβ40 and Aβ42 (e.g. insoluble Aβ40=29±2.3% reduction, p<0.05; insoluble Aβ42=23±1.4% reduction, p<0.05); and 2) significantly reduced plaque number, plaque size, and percent area of the brain covered in plaques (plaque number=13±0.3% reduction, p<0.05; plaque size=16±0.4% reduction, p=0.05; percent area of the brain covered by plaques=14±0.5% reduction, p<0.05; Table 1; FIG. 1F-G). These results are comparable in effect to those of the six month prophylactic studies.

In sum the data show that scyllo-cyclohexanehexol, and to a lesser degree, epi-cyclohexanehexol, can prevent and reverse the AD-like phenotype in TgCRND8 mice, reducing cognitive deficits, amyloid plaques, amyloid angiopathy, Aβ-induced inflammatory response, and accelerated mortality. These effects are likely direct effects of the compounds within the CNS because: 1) the compounds are transported across the blood brain barrier by facilitated transport [17, 19]; and 2) their presence can be demonstrated in the brain tissue of treated mice by gas chromatography-mass spectrometry [20] (data not shown).

There was no change in the levels of APP holoprotein, APP glycosylation, APPs-α or APPs-β, or Aβ speciation (i.e. Aβ 1-38 levels) in brain homogenates from treated and untreated TgCRND8 mice (data not shown). Similarly, the peripheral distribution of Aβ as measured by plasma Aβ42 levels were not different between treated and untreated TgCRND8 mice. Plasma Aβ42 levels in the cohort of TgCRND8 mice following 28 days of cyclohexanehexol therapy at five months of age were: untreated=1144±76 pg/ml; epi-cyclohexanehexol=1079±79 pg/ml; scyllo-cyclohexanehexol=990±73 μg/ml; p=0.87. The absence of alterations in peripheral/plasma Aβ42 may be relevant because plasma Aβ levels were also unchanged in patients who developed a strong antibody response and an apparent clinical improvement following Aβ immuno-therapy [25].

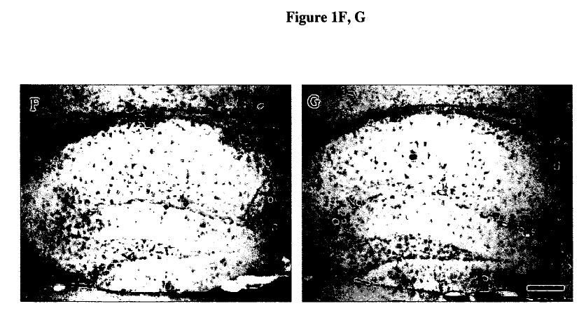

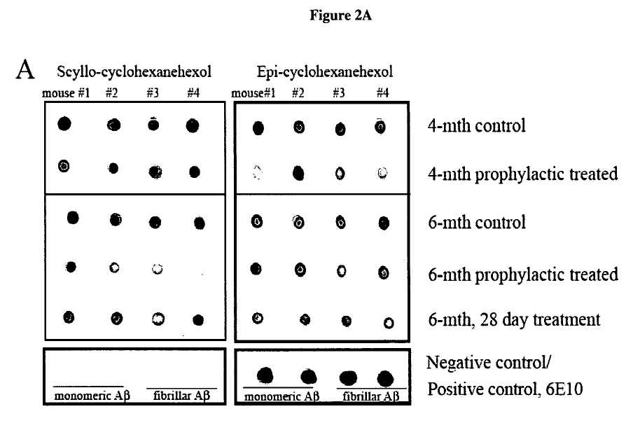

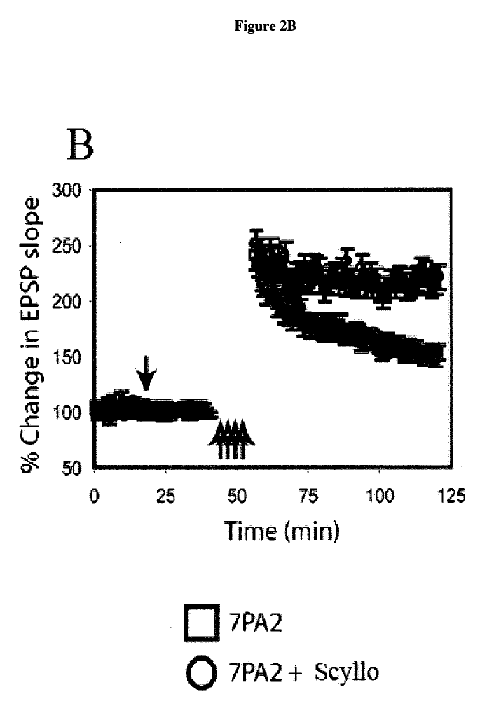

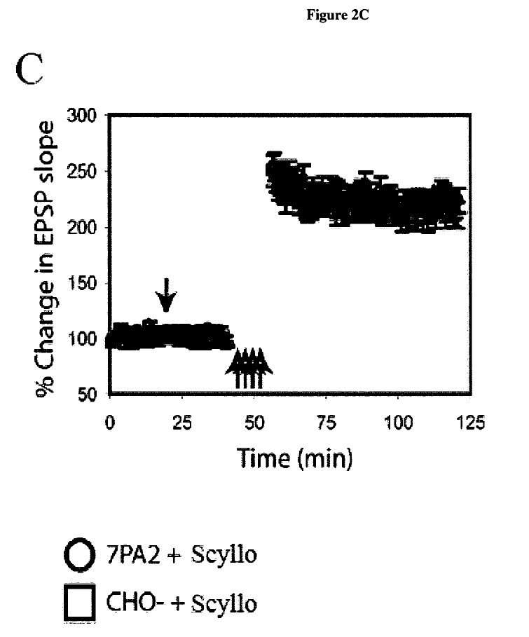

To directly address the possibility that the cyclohexanehexol stereoisomers inhibit Aβ oligomerization in the brain, an activity that they clearly have in vitro [10, 11], a dot blot immunoassay [16] was used to measure levels of AD oligomers in the brains of treated and untreated TgCRND8 mice. This assay employs an antibody that selectively identifies oligomeric Aβ species [16]. The levels of soluble Aβ oligomers were significantly reduced in the brain of treated mice, and these reductions were commensurate with the degree of behavioural and neuropathological improvements induced by these compounds (FIG. 2A). Aβ oligomers were not significantly reduced after one-month treatment with epi-cyclohexanehexol in the five month old TgCRND8 mice with existing pathology (56±4 pixels in untreated TgCRND8 versus 47±2 pixels in epi-cyclohexanehexol treated TgCRND8, p=0.12). Delayed 28-day treatment with scyllo-cyclohexanehexol at five months of age also caused a 30% reduction in soluble Aβ oligomers (63±3 pixels in untreated TgCRND8 versus 45±2 in scyllo-cyclohexanehexol treated TgCRND8, p=0.008). The dot blots were negative for cross-reactivity to tau, α-synuclein and tubulin, demonstrating specificity of the antibody for Aβ in the TgCRND8 brain homogenates. These results directly demonstrate that scyllo-cyclohexanehexol, but not epi-cyclohexanehexol, decreases the amount of soluble Aβ oligomers in the brain.

To address the possibility that scyllo-cyclohexanehexol inhibits Aβ oligomer-induced neurotoxicity, its effects were determined on both long term potentiation (LTP) in mouse hippocampal slices and on synaptic density as measured by the level of synaptophysin immunoreactivity in the brains of TgCRND8 mice. Hippocampal LTP is a measure of synaptic plasticity, and has been shown to be disrupted by natural cell-derived oligomeric Aβ species [42]. As previously reported in rat [42, 43], soluble Aβ oligomers secreted into the conditioned media of CHO cells stably transfected with human APPV717F (7PA2 cells) inhibited LTP in wild-type mouse hippocampal slices (FIG. 2B). However, when the 7PA2-conditioned medium was pretreated in vitro with scyllo-cyclohexanehexol, there was a significant recovery of LTP compared with 7PA2-conditioned media alone (p=0.003; FIG. 2B). Scyllo-cyclohexanehexol had no direct effect on LTP as scyllo-cyclohexanehexol treated culture media from plain CHO cells that were not transfected with human APP (FIG. 2C) and untreated culture media from these cells were indistinguishable from scyllo-cyclohexanehexol treated 7PA2 culture media, i.e., all three samples allowed LTP. The LTP effects were not a result of altered baseline transmission, since scyllo-cyclohexanehexol did not change synaptic response in the absence of a potentiating tetanus (data not shown). In order to correlate this protection of LTP in slice cultures with in vivo effects on synaptic function, the level of synaptophysin immunoreactivity was measured in the CA1 region of the hippocampus in scyllo-cyclohexanehexol-treated and untreated TgCRND8 mice. Synaptophysin immunoreactivity is a measure of synaptic density, which is correlated to synaptic function. The levels of synaptophysin were significantly increased. Thus, scyllo-cyclohexanehexol increased the number of synaptophysin reactive boutons and cell bodies in the CA1 region of the hippocampus by 148% for a prophylactic study group (1610±176/100 μm2 in untreated TgCRND8 mice versus 2384±232/100 μm2 in scyllo-cyclohexanehexol treated TgCRND8 mice; p=0.03) and by 150% for the delayed treatment study (1750±84/100 μm2 in untreated versus 2625±124/100 μm2 in scyllo-cyclohexanehexol treated TgCRND8 mice; p<0.001). Together, the results of the LTP and synaptophysin studies suggest that in the brain, scyllo-cyclohexanehexol may restore the inhibition of LTP induced by naturally secreted human Aβ oligomers, and allow maintenance of synaptic function.

Scyllo-inositol was also administered to TgCRND8 mice for 2 months, ending at 7 months of age. Sustained effects both on cognition and pathology were observed in these treated animals.

### EXAMPLE 2

### Cyclohexanehexol-Based Inhibitors of Ab-Aggregation Prevent and Reverse Alzheimer-Like Features in a Transgenic Model of Alzheimer Disease.

### Summary

When given orally to a transgenic mouse model of Alzheimer disease, cyclohexanehexol stereoisomers inhibit aggregation of amyloid β peptide (Aβ) into high-molecular-weight oligomers in the brain and ameliorate several Alzheimer disease-like phenotypes in these mice, including impaired cognition, altered synaptic physiology, cerebral Aβ pathology and accelerated mortality. These therapeutic effects, which occur regardless of whether the compounds are given before or well after the onset of the Alzheimer disease-like phenotype, support the idea that the accumulation of Aβ oligomers has a central role in the pathogenesis of Alzheimer disease.

To assess their effectiveness in vivo, these compounds were administered to a robust transgenic mouse model of Alzheimer disease [12] (TgCRND8). This model expresses a human amyloid precursor protein transgene (APP695) bearing missense mutations that cause Alzheimer disease in humans (KM670/671NL and V717F). At about 3 months of age, these mice have progressive spatial learning deficits that are accompanied by rising cerebral Aβ levels and by increasing numbers of cerebral amyloid plaques [12]. By 6 months of age, the levels of Aβ and the morphology, density and distribution of amyloid plaques are similar to those seen in brains of people with well-established Alzheimer disease [12]. As in humans with Alzheimer disease, these biochemical, behavioral and neuropathological phenotypes are accompanied by accelerated mortality [12, 13].

TgCRND8 mice and nontransgenic littermates were assigned to sex and age-matched cohorts that were used to test the effectiveness of cyclohexanehexol stereoisomers in two different treatment paradigms. In the first paradigm, cyclohexanehexols were orally administered prophylactically, with treatment beginning at 6 weeks of age (that is, about 6 weeks before onset of phenotype) and continuing until either 4 or 6 months of age. In the second paradigm, compounds were given therapeutically beginning at 5 months of age (when the Alzheimer disease-like phenotype is already well established), and continuing until 6 months of age. Within each of these experimental arms, mice were randomly assigned to receive active compound (1,2,3,4,5/6-(epi) cyclohexanehexol or 1,3,5/2,4,6-(scyllo-) cyclohexanehexol), mock therapy (mannitol, a sugar of similar molecular weight) or no therapy. The endpoints of these studies were: cognitive function (as measured by spatial reference learning in the Morris water maze test [13, 14], brain Aβ levels, neuropathology and mortality. 1,2,3,5/4,6-(myo-) cyclohexanehexol was not included in these studies because prior in vitro studies [11] indicated that myo-cyclohexanehexol was only weakly effective, and because pilot in vivo studies showed no significant benefit (data not shown).

### Methods

The following methods were used in the study:

Mice. Experimental groups of TgCRND8 mice on a C3H/B6 outbred background were initially treated with either epi- or scyllo-cyclohexanehexol. Two cohorts (n=10 mice in each treatment arm) entered the study at 6 weeks of age, and outcomes were analyzed at 4 and 6 months of age. A third cohort of mice (n=10 mice per treatment arm) entered the study at 5 months of age, and outcomes were analyzed after 1 month of treatment. Dose-ranging studies of 0.3 mg/kg/d to 30 mg/d were performed by oral gavage twice daily in mice up to 4 months of age. All experiments were performed according to the Canadian Council on Animal Care guidelines.

Behavioral tests. Morris water maze testing was carried out as previously described [13]. After nonspatial pretraining, mice underwent place discrimination training for 5 d with four trials per day, followed by a cued visible platform trial and a probe trial. An open-field test for motor activity was carried out as described previously [28]. Sensorimotor function was assessed with an Economex accelerating rotarod (Columbus Instruments) as previously described [29].

Cerebral Aβ burden. Brains were removed and fixed one hemisphere from each in 4% paraformaldehyde and embedded in paraffin wax in the midsagittal plane. Sets of sections at 50-mm intervals for analyses (10-14 sections per set) were used. Plaques with primary Aβ-specific antibody (Dako M-0872) were identified and visualized with DAB. AP plaque burden was assessed with Openlab imaging software (Improvision) and used to convert micrographs to binary images for determinations of plaque number and plaque area. Vascular Aβ burden was defined as Aβ plaques originating from or surrounding blood vessels and was analyzed similarly.

Plasma and cerebral Aβ content. Hemi-brain samples were homogenized in a buffered sucrose solution, followed by either a mixture of 0.4% diethylamine and 100 mM NaCl for soluble Aβ levels or cold formic acid for the isolation of total Aβ. After neutralization, samples were diluted and analyzed for Aβ40 and Aβ42 using commercially available kits (Biosource International). Each hemisphere was analyzed in triplicate and the mean±s.e.m. was reported. Western blot analyses were carried out on all fractions using urea gels for Aβ species analyses [30]. Aβ was detected using 6E10 (Biosource International).

Quantification of gliosis. Five randomly selected, evenly spaced sagittal sections from paraformaldehyde-fixed and frozen hemispheres of treated and control mice were collected. Sections were immunolabeled for astrocytes with an antibody against rat GFAβ IgG2a (Dako; diluted 1:50) and for microglia with an antibody to rat CD68 IgG2b (Dako; 1:50). Digital images were captured using a Coolsnap digital camera (Photometrics) mounted to a Zeiss, Axioscope 2 Plus microscope. Images were analyzed using Openlab 3.08 imaging software (Improvision).

Survival census. The probability of survival was assessed using the Kaplan-Meier technique [31], computing the probability of survival at every occurrence of death, thus making it suitable for small sample sizes. For the analyses of survival, 35 mice were used for each treatment group. The Tarone-Ware test was used to assess effects of treatments.

Analysis of APP processing. Mouse hemi-brain samples were homogenized and spun at 109,000 g, in 20 mM Tris pH 7.4, 0.25 M sucrose, 1 mM EDTA and 1 mM EGTA, and a protease inhibitor cocktail, mixed with a mixture of 0.4% DEA (diethylamine) and 100 mMNaCl. The supernatants were analyzed for APP levels by western blotting using the monoclonal antibody 22C11, and the pellets were analyzed for APP holoprotein with the monoclonal antibody C1/6.1 as previously described [32]. After swAPP stable HEK293 cells were treated (or untreated) with scyllo-cyclohexanehexol or 10 nM compound E, a γ-secretase inhibitor, conditioned media was collected for Aβ assay by ELISA, and cell membranes were used for detection of full length APP, the C-terminal fragment of APP and generation of ε-stubs in vitro (that is, incubation at 37° C. for 1 h). The target protein was probed with polyclonal APP C-terminal fragment-specific antibody (Sigma) by western blotting [32].

Soluble Aβ oligomer analyses. Levels of soluble Aβ oligomers were measured by a dot-blot assay with oligomer-specific antibodies on all brain homogenates from all experimental groups [25]. Soluble oligomers were also examined by western blot analyses.

Synaptophysin quantification. Immunohistochemical staining was carried out for synaptophysin on three evenly spaced sagittal sections of paraformaldehyde-fixed treated and control mice. Sections were immunolabeled for synaptophysin with synaptophysin-specific IgG (1:40; Roche). Digital images were captured and analyzed as described above. Within each section, three randomly chosen 100 μm2 areas of the CA1 region of the hippocampus were counted for synaptophysin-reactive cell bodies and boutons. The results are expressed as the mean of the number of reactive bodies and boutons per 100 μm2 [33, 34].

### Supplementary Methods:

Mice. Experimental groups of TgCRND8 mice on a C3H/B6 outbred background were initially treated with either epi- or scyllo-cyclohexanehexol 30 mg/day. This initial dosage was chosen based upon the dosage of myo-cyclohexanehexol (6-18 grams/day/adult or 86-257 mg/kg/day) that is typically administered to human patients for various psychiatric disorders [35]. In these dosages, myo-cyclohexanehexol had no toxicity in humans or animals. The studies described here were also repeated using doses of 5 mg/kg/day −30 mg/day. Two cohorts (n=10 mice in each treatment arm) entered the study at six weeks of age and outcomes were analyzed at four and six months of age. A third cohort of animals (n=10 mice per treatment arm) entered the study at five months of age, and outcomes were then analyzed after one month of treatment. The body weight, coat characteristics and in-cage behaviour was monitored. Mannitol was used as a negative control for potential alterations in caloric intake. All experiments were performed according to the Canadian Council on Animal Care guidelines and approved by the Animal Care Committee, University of Toronto.

Behavioural tests. Morris Water Maze testing was performed as previously described [13]. After non-spatial pre-training, mice underwent place discrimination training for 5 days with 4-trials per day, followed by a cued visible platform to rule out general motivational, learning deficits and motor problems, and a probe trial to evaluate memory. The probe trial was conducted at 72 h after the last testing, and used an annulus-crossing index to assess spatial recall. The annulus-crossing index measures the number of passes over the original platform position relative to passes over the other three quadrants. Data were subjected to a repeated measures analysis of variance (ANOVA) with treatment (untreated, epi- or scyllo-cyclohexanehexol) and genotype (TgCRND8 versus non-Tg) as ‘between-subject’ factors. Open field test for motor activity was performed as described previously

Duration of walking, pausing and grooming were analyzed as indices of spontaneous locomotor activity. Sensorimotor function was examined with an Economex™ accelerating rotarod (Columbus Instruments, Columbus, Ohio), as described elsewhere [29]. The rod was set to accelerate at a rate of 0.2 r.p.m./s, from an initial, constant speed of 5 r.p.m. Latency to fall was recorded in four daily trials, conducted at 30 min intervals. All mice were trained for seven days before testing. The test day performance score for each animal was obtained by summing its latency to fall over the four trials.

Cerebral Aβ burden. Brains were removed and one hemisphere was fixed in 4% paraformaldehyde and embedded in paraffin wax in the mid sagittal plane. To generate sets of systematic uniform random sections, 5 μm serial sections were collected across the entire hemisphere. Sets of sections at 50 μm intervals were used for analyses (10-14 sections/set). Plaques were identified after antigen retrieval with formic acid, and incubation with primary anti-Aβ antibody (Dako M-0872), followed by secondary antibody (Dako StreptABCcomplex/horseradish kit). End products were visualized with DAB and were counter-stained with luxol fast blue. Aβ plaque burden was assessed with Leco IA-3001 image analysis software interfaced with Leica microscope and Hitachi KP-M1U CCD video camera. Openlab imaging software (Improvision, Lexington, Mass.) was then used to convert micrographs to binary images for plaque number and plaque area determinations. Vascular Aβ burden was defined as Aβ plaques originating from or surrounding blood vessels and was analyzed similarly.

Soluble Aβ oligomer Analyses. The levels of soluble Aβ oligomers were measured by a dot blot assay with anti-oligomer specific antibodies on all brain homogenates from all experimental groups [16]. Oligomers were solubilised from one hemi-brain in PBS in the presence of protease inhibitor cocktail (Sigma). After centrifugation at 78,500×g for 1 h at 4° C., the supernatants were analyzed. Protein content was determined by the BCA protein assay (Pierce). Two μg of total protein was spotted onto nitrocellulose, blocked with 10% non-fat milk in TBS before incubation with the biotinylated oligomeric specific antibody. Blots were incubated with streptavidin-HRP and ECL chemiluminescence kit. Soluble and fibrillar Aβ42 were used as negative controls and synthetic oligomeric Aβ42 was used as a positive control. Control samples were re-identified after oligomeric antibody was stripped and re-probing with the anti-Aβ antibody 6E10.

Right hemispheres from 4 month old CRND8 Tg mice treated or untreated with scyllo-cyclohexanehexol were sonicated in 10 vol/wet weight Tris buffered saline (TBS; 20 mM Tris [pH 7.3], 140 mM NaCl containing a protease inhibitor cocktail). Samples were centrifuged (100,000 g, 20 min, 4° C.), supernatant collected and frozen at −80° C. until use. Proteins (20 μg) were separated by SDS-PAGE on a 10-20% Tris-Tricine gel, transferred to a nitrocellulose membrane, blocked for 1 h at room temp with 8% non-fat milk and incubated overnight with anti-Aβ (6E10; 1:2,500). Membranes were rinsed with TBST, exposed to anti-mouse (1:5,000) for 1 h at room temperature, washed with TBST (6×10 min) and developed using enhanced chemiluminescence. Blots were stripped and re-probed with either anti-APP (22C11; 1:1,000) or anti-GAPDH (1:10,000) to confirm equal protein loading.

### Results

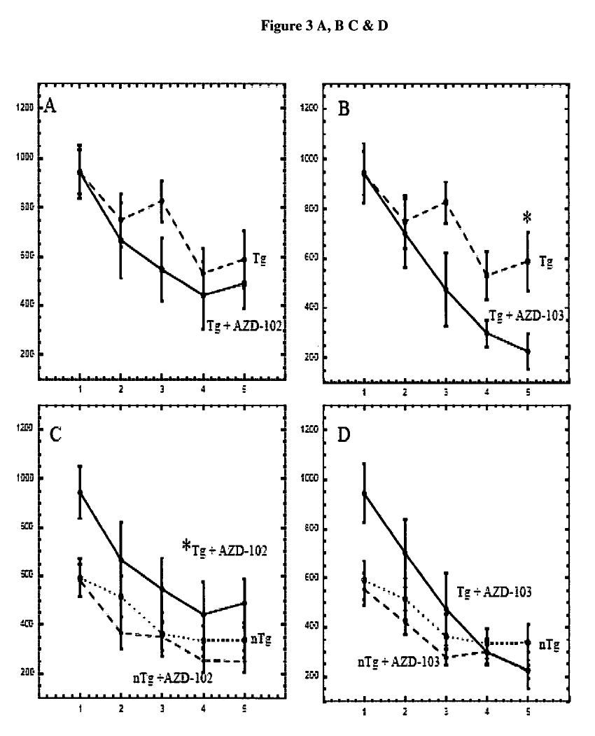

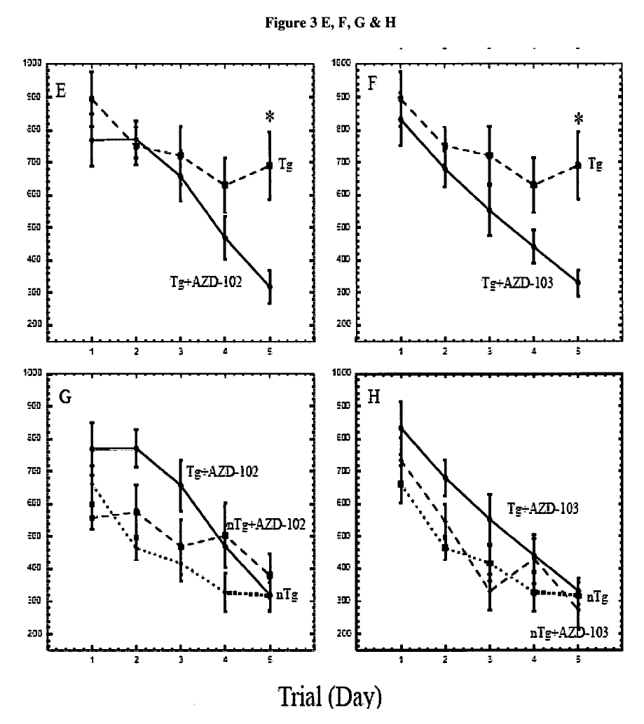

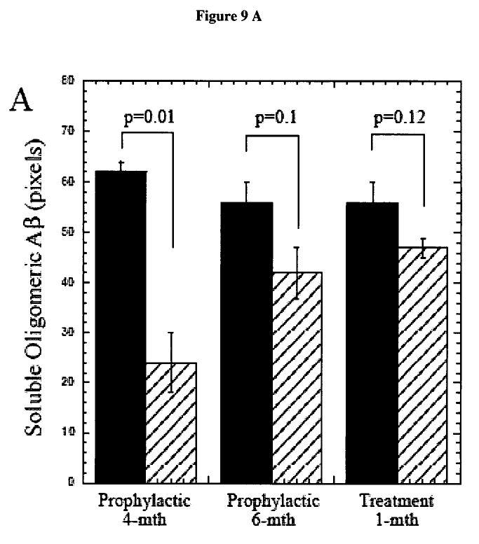

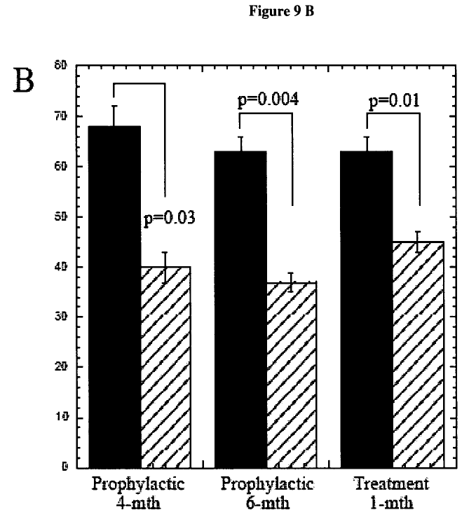

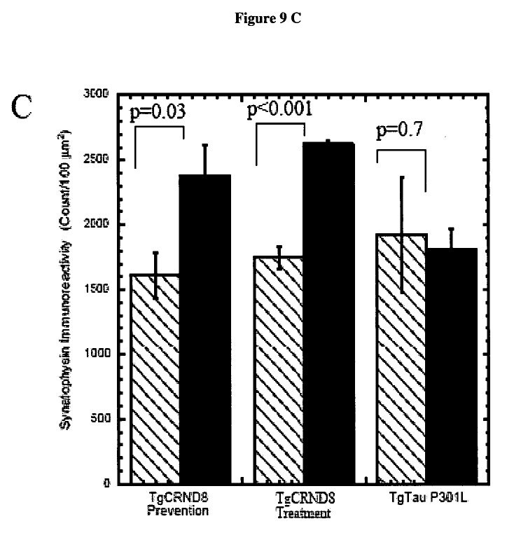

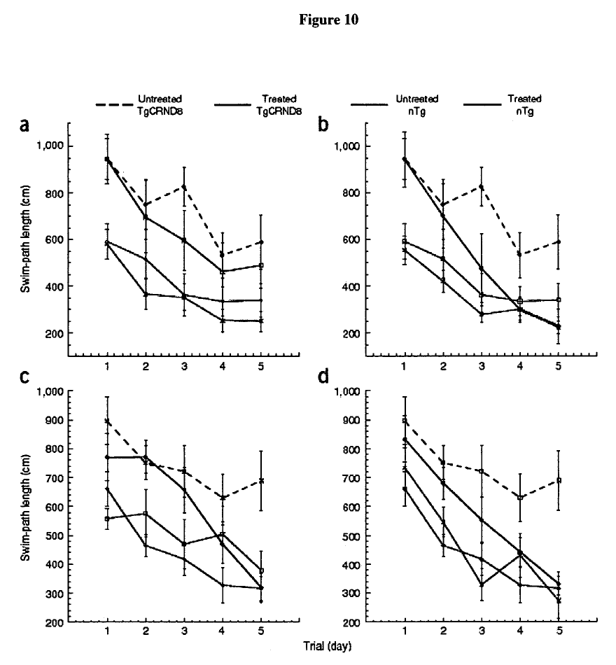

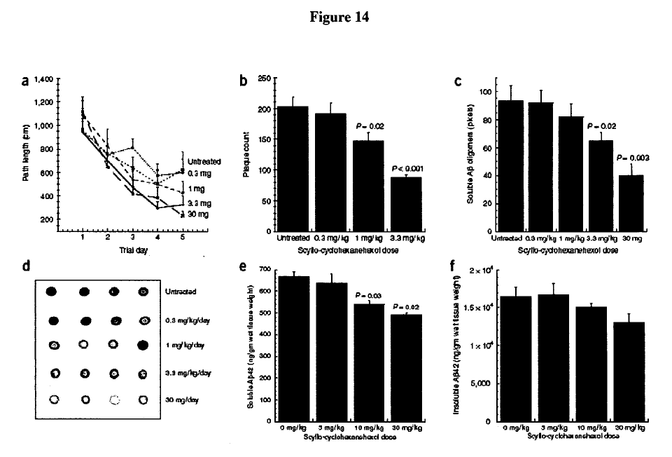

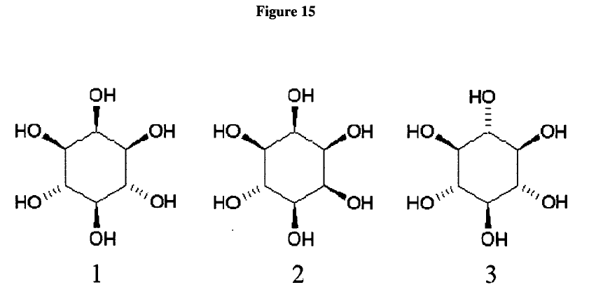

Cyclohexanehexols prevent Alzheimer disease-like phenotype. In the prophylactic trial, TgCRND8 mice were treated with cyclohexanehexol stereoisomers (FIG. 15) from 6 weeks of age until either 4 or 6 months of age. In the cohort examined at 4 months of age, TgCRND8 mice treated with either epi-cyclohexanehexol or scyllo-cyclohexanehexol had significant behavioral improvement compared to untreated TgCRND8 mice (treatment effect, P<0.05; training day effects, P<0.0001; FIG. 3, FIG. 10a, b and Table 3). Scyllo-cyclohexanehexol-treated TgCRND8 mice were indistinguishable from nontransgenic littermates on days 4 and 5 of testing (treatment effect, P=0.97; FIG. 10b). These results were confirmed in a second cohort treated from 6 weeks until 6 months of age. Cyclohexanehexol-treated TgCRND8 mice again had significantly better cognitive function compared to untreated TgCRND8 littermates (P<0.02; FIG. 10c, d), and their performance was indistinguishable from nontransgenic littermates (treatment effects, P=0.14, FIG. 10c and P=0.84, FIG. 10d; epi- and scyllo-cyclohexanehexol, respectively).

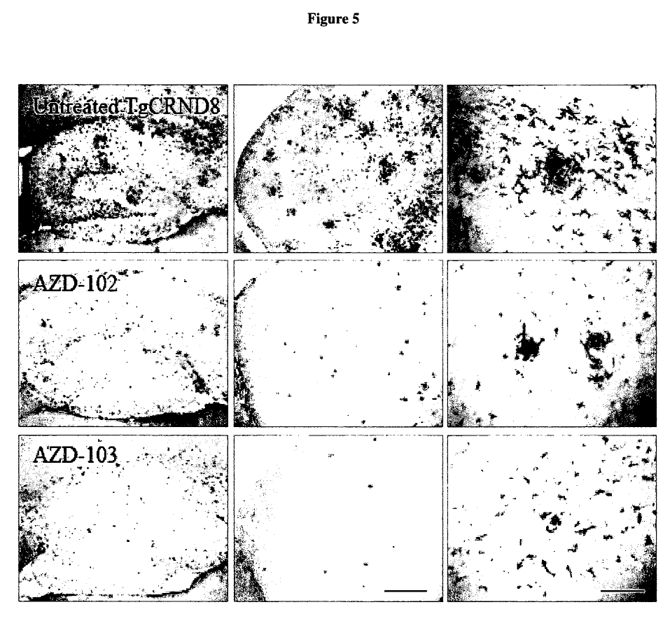

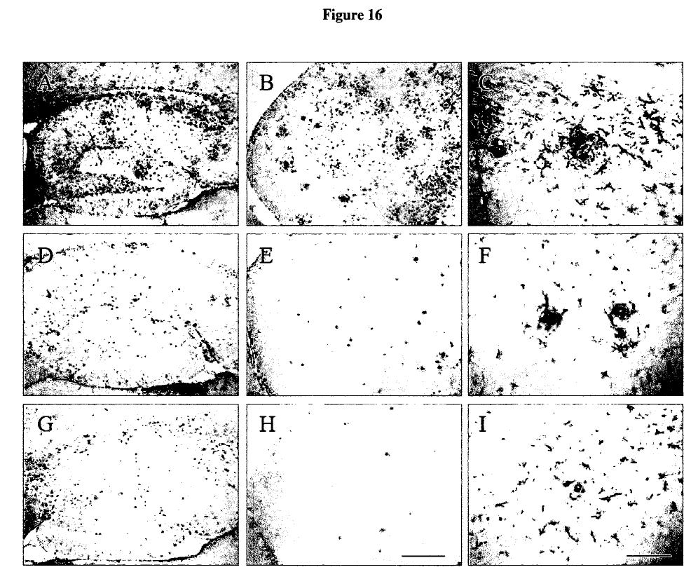

This improvement in cognitive function could not be attributed to nonspecific effects on cognition: cyclohexanehexol treatment had no effect on the performance of nontransgenic mice at any age (treatment effect, P=0.26 and 0.45, respectively; FIG. 10 and Table 3). The improved performance could also not be attributed to nutritional or caloric effects because body weight, activity and coat condition were not different between treated and untreated cohorts. Furthermore, treatment with mannitol had no effect on behavior (P=0.84). Gender effects were not significantly different between any treatment groups (P=0.85, FIG. 5, FIG. 16). Visual acuity was not altered by treatment; all cohorts of mice performed equally well in a cue test (P=0.78).

These behavioral improvements were accompanied by improvements in other Alzheimer disease-like phenotypes including reductions in brain Aβ levels, brain amyloid pathology, synaptic loss, glial inflammatory reaction and reductions in mortality. Like the behavioral improvements described above, however, these changes showed stereoisomer-specific differences, with scyllo-cyclohexanehexol inducing a larger and more sustained effect. Thus, whereas prophylactic treatment with epi-cyclohexanehexol reduced both Aβ40 and Aβ42 at 4 months of age (22-62% reduction; P=0.02; Table 2), by 6 months of age, the concentrations of soluble Aβ40, insoluble Aβ40 and soluble Aβ42 rose to levels equivalent to those observed in untreated TgCRND8 mice (Table 2). In contrast, prophylactic treatment with scyllo-cyclohexanehexol caused a sustained decrease in brain concentrations of both Aβ40 and Aβ42 at both 4 and 6 months of age (2044% reduction; P=0.04 and P=0.02, respectively; Table 2).

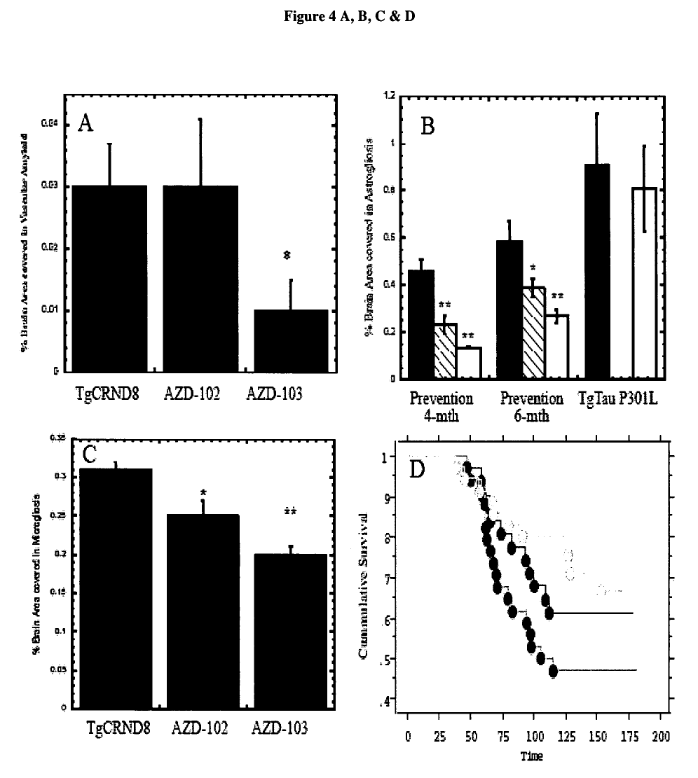

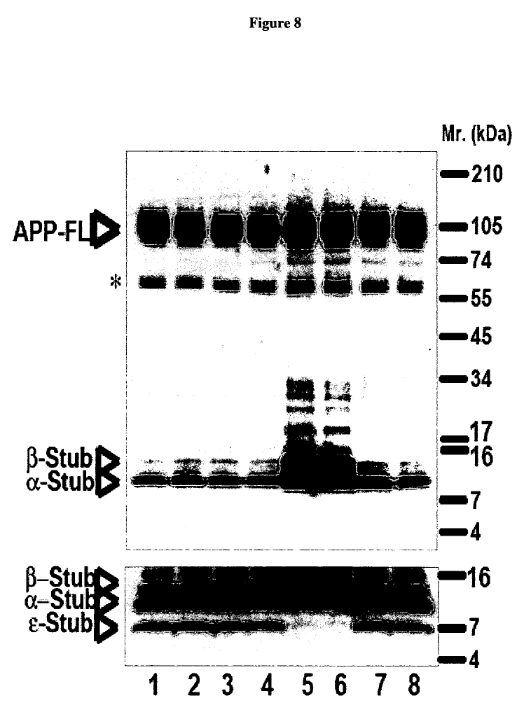

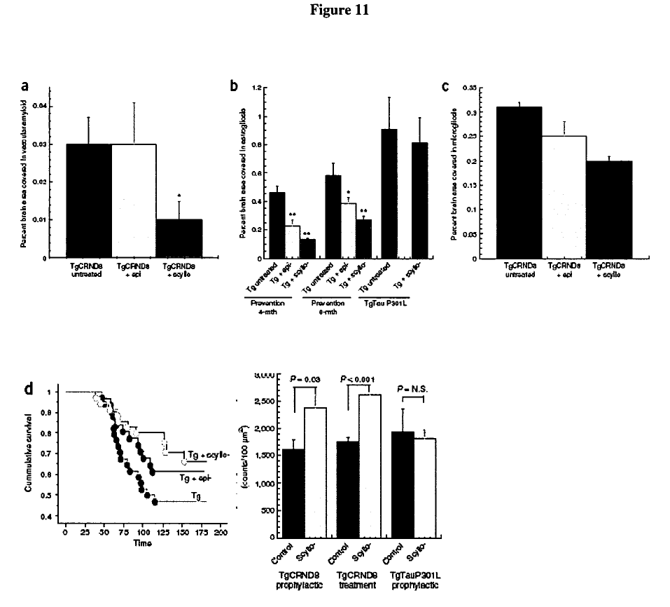

The cyclohexanehexol stereoisomer-induced changes in brain Aβ were accompanied by similar reductions in Alzheimer disease-like neuropathology (plaque burden, angiopathy, glial reaction and synaptic loss; Table 2 and FIG. 4, FIG. 8c, FIG. 11). Compared with untreated TgCRND8 mice, TgCRND8 mice treated prophylactically with epi-cyclohexanehexol showed a significant decrease in mean plaque size at 4 months (P=0.04), but reductions were not sustained at 6 months of age. In contrast, but in agreement with behavioral data, prophylactic treatment with scyllo-cyclohexanehexol caused a sustained decrease in all measures of plaque burden at 4 and 6 months of age (P<0.001; Table 2). Similar differential effects were observed on vascular Aβ deposits (epi-cyclohexanehexol, no change, P=0.87; scyllo-cyclohexanehexol, reduced brain area covered by cerebrovascular Aβ, P=0.05; reduced mean size of cerebrovascular deposits, P=0.008; FIG. 11a).

Astroglial and microglial reactions are a neuropathological feature of both human Alzheimer disease and of transgenic models of Alzheimer disease. Epi-cyclohexanehexol decreased the astrogliotic response at 4 months of age (P=0.002) and at 6 months of age (P=0.04; FIG. 11b), but had no effect on microglial activation (P=0.74 at 6 months of age). In contrast, scyllo-cyclohexanehexol decreased astrogliosis much more efficiently (4 months of age, P<0.0001; 6 months of age, P=0.006), and also attenuated microglial activation (P<0.001; FIG. 11c). This effect is a specific consequence of inhibition of Aβ aggregation, because scyllo-cyclohexanehexol treatment did not prevent the astrogliosis observed in a transgenic TauP301L mouse model of frontotemporal dementia (FIG. 11b).

Like people with Alzheimer disease, TgCRND8 mice have accelerated mortality, and only 50% of untreated TgCRND8 mice survive to 6 months of age (FIG. 11 d). This accelerated mortality was reversed by prophylactic treatment with scyllo-cyclohexanehexol (P=0.02), but not epi-cyclohexanehexol (P=0.34) or mannitol (P=0.89, FIG. 16). Scyllo-cyclohexanehexol had no effect on survival, weight, fur condition or cage behavior of wild-type mice.

Synaptic pruning is a feature of both human Alzheimer disease and of the Alzheimer disease-like illness in many transgenic models. This presumably reflects the chronic synaptotoxic effects of Aβ oligomers, and probably represents a morphological correlate of the cognitive impairment in these models. Measurement of synaptophysin levels in the CA1 region of the hippocampus showed that scyllo-cyclohexanehexol increased the number of synaptophysin-reactive boutons and cell bodies by 148% in the 6-month prophylactic study (P=0.03, FIG. 11e). Again, this effect is likely to arise specifically from its ability to block Aβ oligomer-induced synaptic toxicity, because scyllocyclohexanehexol had no effect on synaptic loss in the transgenic TauP301L mouse model (P=0.89, FIG. 11 e).

### Cyclohexanehexols Reverse Established Disease

To assess whether cyclohexanehexols could abrogate a well-established Alzheimer disease-like phenotype, the start of treatment in TgCRND8 mice was delayed until 5 months of age. At this age, TgCRND8 mice have considerable behavioral deficits, accompanied by profuse Aβ peptide and plaque burdens [12]. Cohorts of TgCRND8 and nontransgenic littermates were either treated for 28 d with epi-cyclohexanehexol or scyllo-cyclohexanehexol, or left untreated. The dosage and oral administration of compounds and the neurochemical and neuropathological assays were as used in the prophylactic experiments. Mortality curves were not generated because the short treatment interval generated too few events.

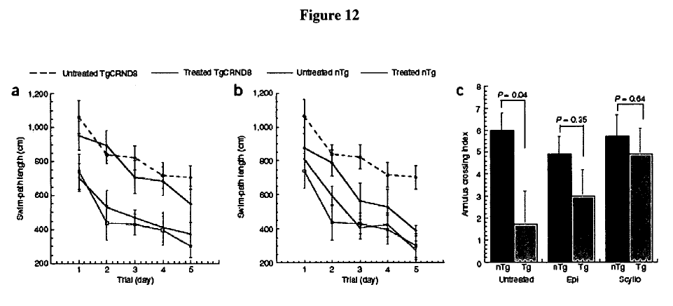

As would be expected from the ineffectiveness of epi-cyclohexanehexol at 6 months in the prophylactic trial, epi-cyclohexanehexol was also ineffective at reversing behavioral deficits in TgCRND8 mice when given from 5 to 6 months of age (spatial learning compared to untreated TgCRND8, P=0.27; compared to nontransgenic littermates, P=0.004; FIG. 12a; spatial reference memory on probe trial using the annulus-crossing index was indistinguishable from untreated TgCRND8 mice, P=0.52; FIG. 12c). Epi-cyclohexanehexol also had no significant effect on levels of Aβ40 or Aβ42 in the brain, percent area of the brain covered with plaques or plaque number in mice with preexisting disease (Table 2).

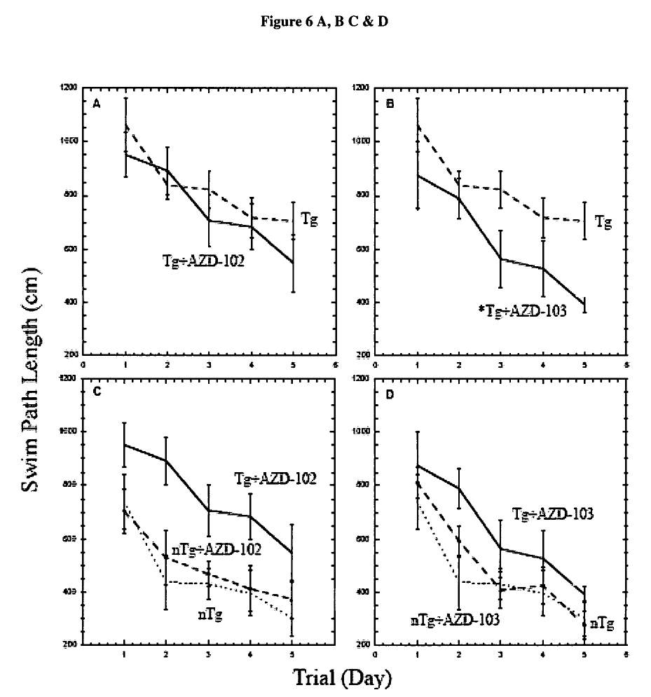

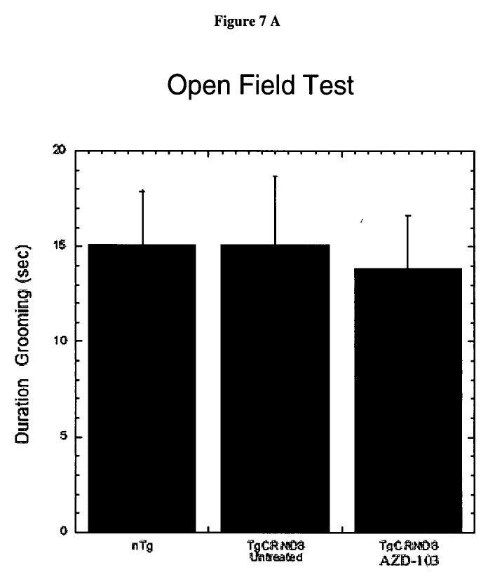

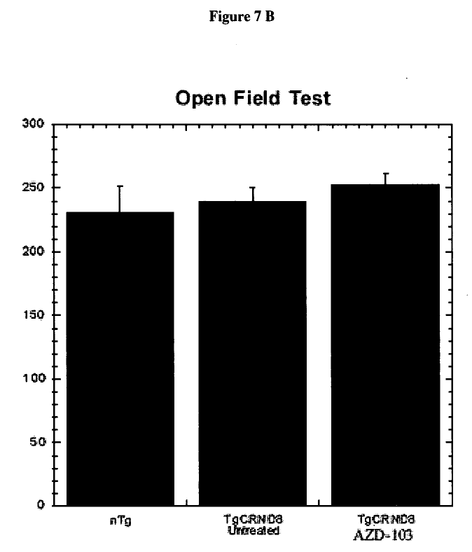

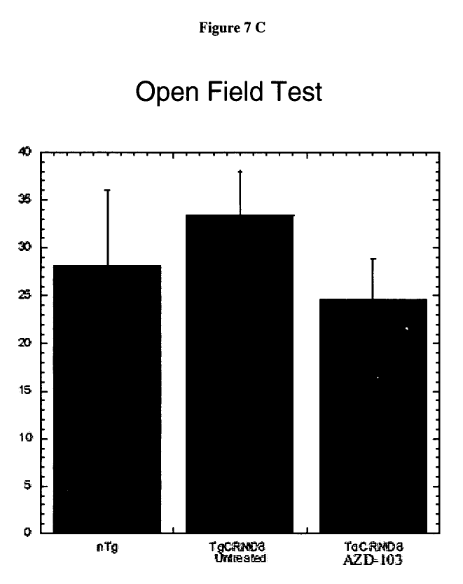

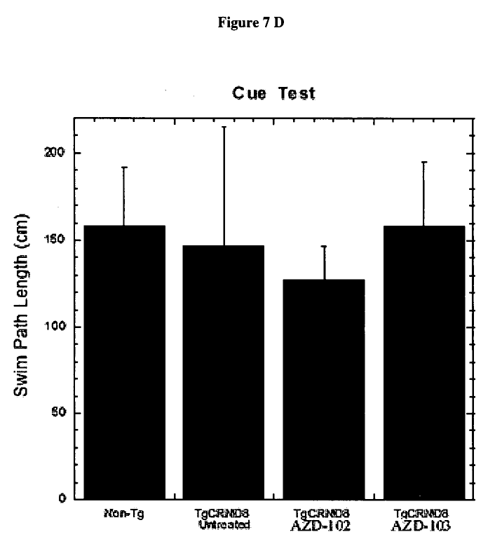

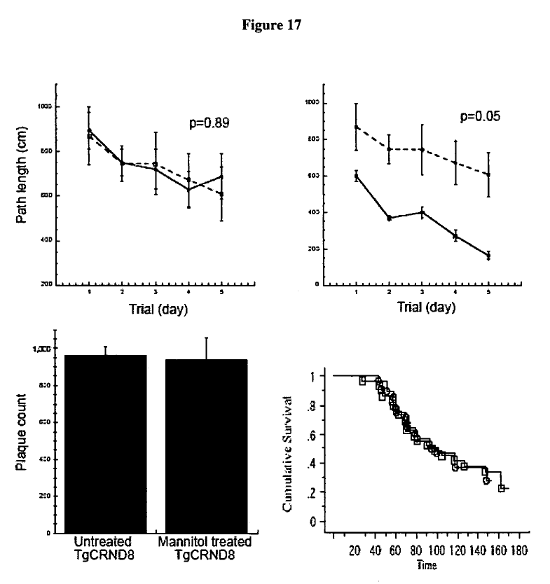

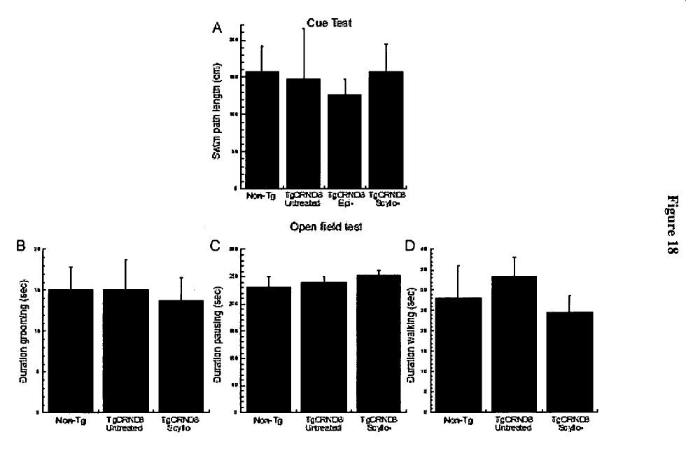

In contrast, but in agreement with results from the prophylactic trial, 28-d treatment of 5-month-old TgCRND8 mice with scyllocyclohexanehexol resulted in improved behavioral performance (treatment effect compared to untreated TgCRND8 mice, P=0.01, Table 3) that was indistinguishable from nontransgenic littermates (P=0.11; FIG. 7, FIG. 12b, FIG. 18). Spatial reference memory, as measured by the annulus-crossing index in the probe trial, showed that scyllo-cyclohexanehexol-treated TgCRND8 mice were indistinguishable from treated or untreated nontransgenic littermates (P=0.64; FIG. 12c, Table 3). This behavioral improvement was accompanied by reduced levels of insoluble Aβ40 and Aβ42 in the brain (for example, insoluble Aβ40, P<0.05; insoluble Aβ42, P<0.05), and significantly reduced plaque number (P<0.05), plaque size (P=0.05) and percent area of the brain covered in plaques (P<0.05; Table 2). These results are comparable in effect size to those of the 6-month prophylactic studies. The 28-d cyclohexanehexol treatment had no effect on cognition or sensorimotor behavior of nontransgenic mice (open-field test and rotarod test were indistinguishable between scyllo-cyclohexanehexol-treated TgCRND8 mice, untreated TgCRND8 mice (P=0.63) and nontransgenic littermates (P=0.89; FIG. 6, FIG. 17)).

There are three non-mutually exclusive mechanisms by which these beneficial effects might occur. The first and least likely mechanism is that cyclohexanehexol stereoisomers may have altered the expression or proteolytic processing of APP. In vitro assays, however, showed that scyllo-cyclohexanehexol had no effect on β- and γ-secretase enzyme activities (FIG. 9); this stereoisomer also had no effect on the levels of APP holoprotein, APP glycosylation, APPs-α or APPs-β in brain homogenates of treated TgCRND8 mice.

A second possibility is that cyclohexanehexol stereoisomers may have simply reduced plasma Aβ levels, thereby creating a ‘sink effect’ that improved efflux of Aβ from the brain into the plasma. But plasma Aβ42 levels were not different between treated and untreated TgCRND8 mice in either the prophylactic or the delayed-therapy trials (P=0.89 and P=0.87, respectively), arguing against an enhanced peripheral clearance mechanism.

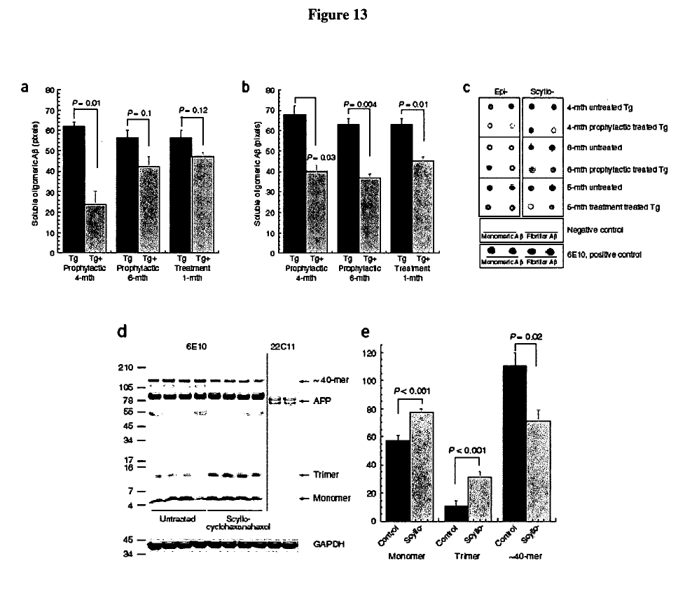

Finally, in light of inhibition of Aβ aggregation and cytotoxicity by cyclohexanehexol in vitro [10, 11], these compounds may directly inhibit Aβ oligomeric assembly and toxicity in the brain and, by precluding oligomeric assembly and insoluble fibril formation [15, 16], may allow Aβ to remain in a biophysical state that can be cleared through normal mechanisms. To address this hypothesis, brain Aβ oligomer levels were measured using a dot-blot immunoassay with an antibody that selectively detects oligomeric Aβ species with a mass greater than 40 kDa16. As predicted, levels of soluble Aβ oligomers were significantly reduced in the brain of cyclohexanehexol-treated TgCRND8 mice and paralleled the degree of behavioral and neuropathological improvements induced by each compound (FIG. 2A, FIG. 8a-b, FIG. 13a-c). Thus, epi-cyclohexanehexol, which was effective only when given prophylactically, reduced Aβ oligomers by 61% at 4 months of age (P=0.01). This effect was not sustained, however, in either the 6-month prophylactic trial (P=0.1) or the 1-month treatment trial (P=0.12). In contrast, treatment with scyllo-cyclohexanehexol induced a 40% reduction in soluble Aβ oligomers in both the 4- and 6-month prophylactic trials (P=0.03 and P=0.004, respectively), and caused a 30% reduction in the 28-d treatment trial (P=0.008).

To investigate which species of Aβ oligomers were principally affected in the brain of scyllo-cyclohexanehexol-treated mice, western blots of soluble fractions of brain homogenates from 4-month-old treated and untreated TgCRND8 mice were performed. These experiments showed that scyllo-cyclohexanehexol reduced the abundance of high-molecular-weight Aβ oligomeric species by 25±4.5% (P=0.02; FIG. 13d,e), and caused a concomitant 282±5% and 133±4% increase in low-molecular-weight trimeric (P<0.001) and monomeric species, respectively (P<0.001). These results suggest that scyllocyclohexanehexol exerts its beneficial effect through inhibition and/or disaggregation of high-molecular-weight Aβ oligomers.

Finally, dose-response studies, in which TgCRND8 mice treated from 6 weeks to 4 months of age with scyllo-cyclohexanehexol (0.3-30 mg/kg/d), showed a dose-dependent improvement in spatial memory (P<0.05; FIG. 14a), which was accompanied by a corresponding dose-dependent decrease in both plaque count (P<0.02; FIG. 14b) and in brain Aβ oligomers (P<0.02; FIG. 14c, d). These effects were maintained at 6 months of age, as shown by the dose-dependent decrease in soluble Aβ42 levels within the brain (P<0.02; FIG. 14e, f, Table 2).

### Discussion

The results reported here show that orally administered scyllocyclohexanehexol and, to a lesser extent, epi-cyclohexanehexol significantly inhibit Alzheimer disease-like behavioral deficits, neuropathology and accelerated mortality in the TgCRND8 mouse model of Alzheimer disease. Notably, these effects occur regardless of whether the compounds are given during the latent (or presymptomatic) phase or the overt (or symptomatic) phase of the Alzheimer disease-like illness.

Several independent lines of evidence suggest that the beneficial effects of cyclohexanehexol stereoisomers arise from a decrease in the accumulation of neurotoxic Aβ oligomers in brain. First, the compounds are transported across the blood-brain barrier by facilitated transport [17-20]; and their presence in brain tissue of treated mice can be determined by gas chromatography-mass spectrometry [20]. Second, the stereoisomer-specific effects on brain Aβ oligomers levels, cognitive deficits, amyloid neuropathology and accelerated mortality in the TgCRND8 mouse model of Alzheimer diseasegenerally parallel the relative effects on blocking Aβ oligomerization and cytotoxicity in vitro [10, 11]. Third, there is a close correlation between the dose-response curves for the effect of scyllo-cyclohexanehexol on brain oligomer levels and on cognition, neuropathology and total brain Aβ levels. Finally, consistent with the possibility that cyclohexanehexols and Aβ-specific antibodies (especially those directed to the N-terminal residues of Aβ [21-23]) both have antiaggregation effects, the behavioral and neuropathological effects of cyclohexanehexol therapy are similar to those previously shown by Aβ-specific antibodies in this model [13]. They also resemble the beneficial effects described in preliminary human clinical trials of Aβ42 immunization [24, 25].

Oligomeric aggregates of misfolded proteins is an important mechanism underlying neurodegenerative disease. This concept necessarily predicts that attempts to block the formation of toxic oligomeric aggregates should also block the disease phenotype, as was shown here. This is supported by the observation that oral administration of another low-molecular-weight osmolyte, trehalose, decreased polyglutamine aggregates in brain, improved motor dysfunction and extended lifespan of a mouse model of Huntington disease [26]. Trehalose, cyclohexanehexols and other low-molecular-weight osmolytes may function by modulating the folding of proteins that are prone to misfold and aggregate into toxic oligomers [9]. By stabilizing these proteins as monomers and nontoxic conformers that are unable to further assemble (for example, P-sheet spherical micelles [10, 11]), these compounds would: (i) favor the disassembly of high-molecular-weight oligomers and fibrillar deposits; (ii) block the clinical consequences; and (iii) by maintaining the protein in a soluble state, encourage its clearance by normal pathways. There is debate as to which species of Aβ are neurotoxic. The data suggests that these toxic species are likely to be higher-order oligomers. This conclusion is in general agreement with the recent observation that the abundance of a high-molecular-weight 12-unit oligomer (Aβ*) correlates with the appearance of neurotoxicity in the TgAPPswe mouse [27]. It is presently unclear whether the scyllo-cyclohexanehexol-sensitive ˜40-mer peptide and Aβ* are related species (for example, as a multimer of Aβ*), whether these different apparent molecular weights reflect the existence of multiple toxic Aβ oligomer species or whether the different apparent molecular masses simply reflect differences in sample preparation and electrophoretic procedures.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant invention.

TABLE 1
Cyclohexanehexol treatment for 28 days decreases brain
Aβ40 and Aβ42 Levels and amyloid plaques at 6 months of age.

| | Aβ40 | | | | | | Mean |
|---|---|---|---|---|---|---|---|
| | (ng/gm | | Aβ42 | | Total | Total | Plaque |
| | wet brain ± sem) | | (ng/gm wet brain ± sem) | | Plaque | Plaque | Size |
| | Soluble | Insoluble | Soluble | Insoluble | Count | Area (μm²) | (μm²) |
| Control | 204 ± 4 | 4965 ± 457 | 426 ± 14 | 14503 ± 1071 | 1441 ± 29 | 486002 ± 16156 | 401 ± 14 |
| Epi- | 264 ± 11 | 3637 ± 113* | 540 ± 14 | 12830 ± 330 | 1342 ± 114 | 459706 ± 49966 | 346 ± 6 |
| cyclohexanchexol | | | | | | | |
| Scyllo- | 178 ± 11 | 3527 ± 241* | 374 ± 23 | 11115 ± 647* | 1260 ± 27* | 420027 ± 14986* | 336 ± 6* |
| cyclohexanehexol | | | | | | | |

ANOVA with Fisher's PLSD,
*p < 0.05.

TABLE 2
Prophylactic and treatment with Cyclohexanehexol decreases brain
Aβ40 and Aβ42 Levels and Plaques

| Aβ40 | | | | | |
|---|---|---|---|---|---|
| (ng/gm | | Aβ42 | | Total | Mean |
| wet brain ± sem) | | (ng/gm wet brain ± sem) | | Plaque | Plaque |
| Soluble | Insoluble | Soluble | Insoluble | Area (μm²) | Size (μm²) |

4-month Prophylactic

| | | | | | | |
|---|---|---|---|---|---|---|
| Control | 75 ± 6 | 1163 ± 9 | 273 ± 18 | 5658 ± 248 | 100766 ± 7564 | 136 ± 15 |
| Epi-cyclohexanehexol | 43 ± 7* | 615 ± 32† | 85 ± 7† | 4059 ± 179* | 65042 ± 5199 | 95 ± 43 |
| Scyllo-cyclohexanehexol | 37 ± 5* | 437 ± 80† | 206 ± 8* | 4409 ± 135* | 63847 ± 2895 | 103 ± 4* |

6-month Prophylactic

| | | | | | | |
|---|---|---|---|---|---|---|
| Control | 187 ± 29 | 3576 ± 172 | 626 ± 87 | 15802 ± 237 | 41128 ± 11912 | 423 ± 22 |
| Epi-cyclohexanehexol | 188 ± 24 | 3668 ± 149 | 665 ± 39 | 13943 ± 277† | 380456 ± 13498 | 370 ± 9 |
| Scyllo-cyclohexanehexol | 105 ± 8* | 2453 ± 251† | 475 ± 26* | 12588 ± 82† | 262379 ± 5373† | 339 ± 10† |

1 month Treatment

| | | | | | | |
|---|---|---|---|---|---|---|
| Control | 204 ± 4 | 4965 ± 457 | 426 ± 14 | 14503 ± 1071 | 486002 ± 16156 | 401 ± 14 |
| Epi-cyclohexanehexol | 264 ± 11 | 3637 ± 113* | 540 ± 14 | 12830 ± 330 | 459706 ± 49966 | 346 ± 6 |
| Scyllo-cyclohexanehexol | 178 ± 11 | 3527 ± 241* | 374 ± 23 | 11115 ± 647* | 420027 ± 14986* | 336 ± 6* |

ANOVA with Fisher's PLSD,
†p < 0.001 and
*p < 0.05.

TABLE 3
Overall effect of cyclohexanehexols on cognitive function
Repeated measures ANOVA with compound (untreated, epi- and
scyllo-cyclohexanehexol), genotype (Tg versus nTg) as between
subject factors and day of testing as within subject factors.
1. Prophylactic Study:

| | | |
|---|---|---|
| 4-months of age test with | | |
| epi-cyclohexanehexol: | | |
| Epi-cyclohexanehexol effect | F(1,35) = 0.2 | P = 0.89 |
| Genotype effect | F(1, 35) = 42 | P < 0.0001 |
| Day effect | F(4, 140) = 11 | P < 0.0001 |
| Epi-cyclohexanehexol × genotype | F(1, 35) = 1.2 | P = 0.28 |
| Day × epi-cyclohexanehexol | F(4, 140) = 0.4 | P = 0.81 |
| Day × genotype | F(4, 140) = 0.4 | P = 0.4 |
| Epi-cyclohexanehexol × genotype × day | F(4, 140) = 0.62 | P = 0.65 |
| 4-months of age test with scyllo- | | |
| cyclohexanehexol | | |
| Scyllo-cyclohexanehexol effect | F(1, 35) = 8.5 | P = 0.006 |
| Genotype effect | F(1, 35) = 31 | P < 0.0001 |
| Day effect | F(4, 140) = 15.3 | P < 0.0001 |
| Scyllo-cyclohexanehexol × genotype | F(1, 35) = 1.1 | P = 0.3 |
| Day × scyllo-cyclohexanehexol | F(4, 140) = 1.0 | P = 0.44 |
| Day × genotype | F(4, 140) = 1.5 | P = 0.2 |
| Scyllo-cyclohexanehexol × genotype × | F(4, 140) = 0.7 | P = 0.63 |
| day | | |
| 6-months of age test with epi- | | |
| cyclohexanehexol | | |
| Epi-cyclohexanehexol effect | F(1, 51) = 0.9 | P = 0.35 |
| Genotype effect | F(1, 51) = 23 | P < 0.0001 |
| Day effect | F(4, 204) = 13 | P < 0.0001 |
| Epi-cyclohexanehexol × genotype | F(1, 51) = 5.6 | P = 0.02 |
| Day × epi-cyclohexanehexol | F(4, 204) = 2.0 | P = 0.10 |
| Day × genotype | F(4, 204) = 0.6 | P = 0.65 |
| Epi-cyclohexanehexol × genotype × day | F(4, 204) = 1.83 | P = 0.12 |
| 6-months of age test with scyllo- | | |
| cyclohexanehexol | | |
| Scyllo-cyclohexanehexol effect | F(1, 52) = 3.2 | P = 0.08 |
| Genotype effect | F(1, 52) = 25 | P < 0.0001 |
| Day effect | F(4, 208) = 23 | P < 0.0001 |
| Scyllo-cyclohexanehexol × genotype | F(1, 52) = 5.9 | P = 0.02 |
| Day × scyllo-cyclohexanehexol | F(4, 208) = 2.1 | P = 0.08 |
| Day × genotype | F(4, 208) = 0.5 | P = 0.75 |
| Scyllo-cyclohexanehexol × genotype × | F(4, 208) = 0.7 | P = 0.6 |
| day | | |

2. Treatment Study

| | | |
|---|---|---|
| Epi-cyclohexanehexol | | |
| Epi-cyclohexanehexol effect | F(1, 46) = 0.9 | P = 0.94 |
| Genotype effect | F(1, 46) = 18 | P = 0.0004 |
| Day effect | F(4, 184) = 20 | P < 0.0001 |
| Epi-cyclohexanehexol × genotype | F(1, 46) = 2.0 | P = 0.17 |
| Day × epi-cyclohexanehexol | F(4, 184) = 1.7 | P = 0.15 |
| Day × genotype | F(4, 184) = 1.5 | P = 0.22 |
| Epi-cyclohexanehexol × genotype × day | F(4, 184) = 1.06 | P = 0.38 |
| Scyllo-cyclohexanehexol | | |
| Scyllo-cyclohexanehexol effect | F(1, 45) = 0.5 | P = 0.5 |
| Genotype effect | F(1, 45) = 16 | P = 0.0008 |
| Day effect | F(4, 180) = 17 | P < 0.0001 |
| Scyllo-cyclohexanehexol × genotype | F(1, 45) = 1.9 | P = 0.18 |
| Day × scyllo-cyclohexanehexol | F(4, 180) = 0.29 | P = 0.88 |
| Day × genotype | F(4, 180) = 0.29 | P = 0.88 |
| Scyllo-cyclohexanehexol × genotype × | F(4, 180) = 0.7 | P = 0.6 |
| day | | |

### The following are citations for publications.

1. Selkoe, D. J. Deciphering the genesis and fate of amyloid beta-protein yields novel therapies for Alzheimer disease. J. Clin Invest. 110, 1375-1381 (2002).
2. Glabe, C. C. Amyloid accumulation and pathogenesis of Alzheimer's disease: significance of monomeric, oligomeric and fibrillar Aβ. Subcell Biochem. 38, 167-177 (2005).
3. McLaurin, J. & Chakrabartty, A. Membrane disruption by Alzheimer beta-amyloid peptides mediated through specific binding to either phospholipids or gangliosides. Implications for neurotoxicity. J. Biol. Chem. 271, 26482-26489 (1996).
4. McLaurin, J. & Chakrabartty, A. Characterization of the interactions of Alzheimer beta-amyloid peptides with phospholipid membranes. Eur. J. Biochem. 245, 355-363 (1997).
5. Koppaka, V. & Axelson, P. H. Accelerated accumulation of amyloid beta proteins on oxidatively damaged lipid membranes. Biochemistry 39, 10011-10016 (2000).
6. Mizuno, T. et al., Cholesterol-dependent generation of a seeding amyloid beta-protein in cell culture. J. Biol. Chem. 274, 15110-15114 (1999).
7. Choo-Smith, L. P. & Surewicz, W. K. The interaction between Alzheimer amyloid beta(1-40) peptide and ganglioside GM1-containing membranes. FEBS Lett 402, 95-98 (1997).
8. Yanagisawa, K. et al., GM1 ganglioside-bound amyloid beta-protein (A beta): a possible form of preamyloid in Alzheimer's disease. Nat. Med. 1, 1062-1066 (1995).
9. Auton, M, Bolen D W. Predicting the energetics of osmolyte-induced protein folding/unfolding. Proc Natl Acad Sci USA. 102, 15065-15068 (2005).
10. McLaurin, J. Franklin, T. Chakrabartty, A. & Fraser, P. E. Phosphatidylinositol and inositol involvement in Alzheimer amyloid-beta fibril growth and arrest. J. Mol. Biol. 278, 183-194 (1998).
11. McLaurin, J., Goloumb, R., Jurewicz, A., Antel, J. P. & Fraser, P. E. Inositol stereoisomers stabilize an oligomeric aggregate of Alzheimer amyloid beta peptide and inhibit abeta-induced toxicity. J. Biol. Chem. 275, 18495-18502 (2000).
12. Chishti, M. A. et al., Early-onset amyloid deposition and cognitive deficits in transgenic mice expressing a double mutant form of amyloid precursor protein 695. J. Biol Chem 276, 21562-21570 (2001).
13. Janus, C. et al., Aβ peptide immunization reduces behavioural impairment and plaques in a model of Alzheimer's disease. Nature 408, 979-982 (2000).
14. Morris, R. Development of a water-maze procedure for studying spatial learning in the rat. J. Neurosci Methods 11, 47-60 (1984).
15. Klein, W. L. Aβ toxicity in Alzheimer's disease: globular oligomers (ADDLs) as a new vaccine and drug targets. Neurochem. Int. 41, 345-352 (2002).
16. Kayed, R. et al., Common Structure of Soluble amyloid oligomers implies common mechanism of pathogenesis. Science 300, 486-489 (2003).
17. Spector, R. Myo-inositol transport through the blood-brain barrier. Neurochemical Research 13, 785-787 (1988).
18. Uldry, M. et al., Identification of a mammalian H(+)-myo-inositol symporter expressed predominantly in the brain. EMBO 20, 4467-4477 (2001).
19. Uldry, M. & Thorens B. The SLC2 family of facilitated hexose and polyol transporters. Pflugers Acta, 447, 480-489 (2004).
20. Shetty, H. U. & Hollway, H. W. Assay of myo-inositol in cerebrospinal fluid and plasma by chemical ionization mass spectrometry of the hexaacetate derivative. Biol. Mass Spec. 23, 440-444 (1994).
21. McLaurin, J. et al., Therapeutically effective antibodies against amyloid-beta peptide target amyloid-beta residues 4-10 and inhibit cytotoxicity and fibrillogenesis. Nat. Med. 8, 1263-1269 (2002).
22. Frenkel, D., Katz, O., & Solomon, B. Immunization against Alzheimer's β-amyloid plaques via EFRH phage administration. Proc. Natl. Acad. Sci. USA 97, 11455-11459 (2000).
23. Bard, F., et al., Epitope and isotype specificities of antibodies to β-amyloid peptide for protection against disease-like neuropathology. Proc. Natl. Acad. Sci. USA 100,2023-2028 (2003).
24. Nicoll, J. A. R., Wilkinson, D., Holmes, C., Steart, P., Markham, H. & Weller, R. O. Neuropathology of human Alzheimer disease after immunization with amyloid-β peptide: a case report. Nat. Med. 9, 448-452 (2003).
25. Hock, C. et al., Antibodies against beta-amyloid slow cognitive decline in Alzheimer's disease. Neuron 38, 547-554 (2003).
26. Tanaka, M. et al., Trehalose alleviates polyglutamine-mediated pathology in a mouse model of Huntington disease. Nat. Med. 10, 148-154 (2004).
27. Lesne, S., et al., A specific amyloid-β protein assembly in the brain impairs memory. Nature 440, 352-357 (2006).
28. Vaucher, E. et al., Object Recognition Memory and Cholinergic Parameters in Mice Expressing Human Presenilin 1 Transgenes. Exp. Neurol. 175, 398-406 (2002).
29. Mount, H. T. J., Progressive sensorimotor impairment is not associated with reduced dopamine and high energy phosphate donors in a model of ataxia-telangiectasia. J Neurochem 88:1449-1454.
30. Wiltfang, J. et al., Highly conserved and disease-specific patterns of carboxyterminally truncated Abeta peptides 1-37/38/39 in addition to 1-40/42 in Alzheimer's disease and in patients with chronic neuroinflammation J Neurochem 81, 481-496 (2002)
31. Haccou, P., & Mellis, E., Statistical Analysis of Behavioural Data, pg 120-186, Oxford University Press, Oxford (1995).
32. Chen, F. et al. Carboxyl-terminal fragments of Alzheimer beta-amyloid precursor protein accumulate in restricted and unpredicted intracellular compartments in presenilin 1-deficient cells. J Biol. Chem. 275, 36794-36802 (2002).
33. Phinney, A. et al., No hippocampal neuron or synaptic bouton loss in learning-impaired aged β-amyloid precursor protein-null mice. Neuroscience 90, 1207-1216 (1999).
34. Hu, L. et al., The impact of Aβ-plaques on Cortical Cholinergic and Non-cholinergic Presynaptic boutons in Alzheimer's Disease-like Transgenic mice. Neuroscience 121, 421-432 (2003).
35. Levine, J. Controlled trials of inositol in psychiatry. Eur. Neuropsychopharmacology 7, 147-155 (1997).
36. Orgogozo, J. M., et al., Subacute meningoencephalitis in a subset of patients with AD after Aβ42 immunization. Neurology 61, 46-54 (2003).
37. Schenk, D, et al., Immunization with amyloid-beta attenuates Alzheimer-disease-like pathology in the PDAPP mouse. Nature 400, 173-177 (1999).
38. Golde, T. E. Alzheimer disease therapy: Can the amyloid cascade be halted? J. Clin. Invest. 111, 11-18 (2003).
39. Sarvey, J M, Burgard E C & Decker G. Long-term potentiation: studies in the hippocampal slice. J Neurosci Methods 28, 109-124 (1989).
40. Stanton, P K & Sarvey J M. Norepinephrine regulates long-term potentiation of both the population spike and dendritic EPSP in hippocampal dentate gyrus. Brain Res Bull. 18, 115-119 (1987).
41. Moyer, J R Jr, & Brown T H. Methods for whole-cell recording from visually preselected neurons of perirhinal cortex in brain slices from young and aging rats. J Neurosci Methods. 86, 35-54 (1998).
42. Walsh, D. M. et al., Naturally secreted oligomers of amyloid beta protein potently inhibit hippocampal long-term potentiation in vivo. Nature 416, 535-539 (2002a)
43. Walsh, D. M., Klyubin, I., Fadeeva, J. V., Rowan, M. J. & Selkoe, D. J. Amyloid-beta oligomers: their production, toxicity and therapeutic inhibition. Biochem Soc. Trans. 30, 552-557 (2002b).

What is claimed is:

1. A pharmaceutical composition comprising one or more scyllo-inositol compound of the formula Ia or Ib 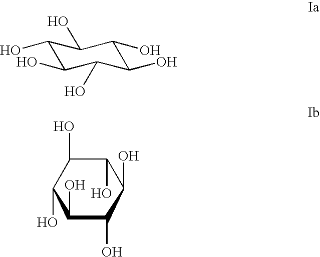
or a compound of the formula Ia or Ib wherein one, two or three hydroxyl groups are replaced by substituents with retention of configuration, or pharmaceutically acceptable salts thereof, in a therapeutically effective amount to provide beneficial effects in the treatment of an amyloid-related disease, and a pharmaceutically acceptable carrier, excipient or vehicle.

2. A pharmaceutical composition of claim 1 comprising a compound of the formula Ia or Ib wherein one, two or three hydroxyl groups are replaced with hydrogen, alkyl, acyl, alkenyl, alkoxy, ═O, cycloalkyl, halogen, —NHR¹ wherein R¹ is hydrogen, acyl, alkyl or —R²R³ wherein R² and R³ are the same or different and represent acyl or alkyl; —PO₃H₂; —SR⁴ wherein R⁴ is hydrogen, alkyl, or —O₃H; and —OR³ wherein R³ is hydrogen, alkyl, or —SO₃H.

3. A pharmaceutical composition of claim 1 comprising a compound of the formula Ia or Ib wherein one or more of the hydroxyl groups are replaced with C₁-C₆ alkyl, C₂-C₆ alkenyl, C₁-C₆alkoxy, C₃-C₁₀cycloalkyl, C₁-C₆ acyl, —NH₂, —NHR¹, —NR²R³, halo, haloalkyl, haloalkoxy, hydroxyalkyl, or oxo.

4. A pharmaceutical composition according to claim 1 wherein the beneficial effect is a reduction in total vascular load, a reduction in astrogliosis and/or a reduction in microgliosis.

5. A pharmaceutical composition according to claim 1 wherein the amyloid-related disease is Alzheimer's disease.

6. A pharmaceutical composition according to claim 1 wherein the amyloid-related disease is dementia.

7. A pharmaceutical composition according to claim 1 wherein the amyloid-related disease is mild cognitive impairment

8. A pharmaceutical composition according to claim 5 for oral administration.

9. A pharmaceutical composition according to claim 1 wherein the compound of the formula Ia or IB is in a therapeutically effective amount which improves cognitive function, reduces vascular load, reduces astrogliosis, reduces amyloid burden, and/or reduces microgliosis.

10. A pharmaceutical composition according to claim 9, wherein the therapeutically effective amount is about 1 mg to about 200 mg per kg per day, about 100 mg per kg per day or about 1 mg to about 50 mg per kg per day.

11. A pharmaceutical composition according to claim 1 wherein the compound of the formula Ia or Ib is produced using microbial process steps.

12. A method for treating an amyloid-related disease in a subject comprising administering to a subject a therapeutically effective amount of a pharmaceutical composition according to claim 1.

13. A method of delaying the progression of an amyloid-related disease in a subject comprising administering to the subject a therapeutically effective amount of a pharmaceutical composition as claimed in claim 1.

14. A method for reducing one or more of vascular load, astrogliosis, and/or microgliosis in a subject comprising administering a therapeutically effective amount of a pharmaceutical composition as claimed in claim 1.

15. A method according to claim 14 wherein the therapeutically effective amount of a compound of the formula Ia or Ib is about 1 mg to about 200 mg per kg per day.

16. A method according to claim 14 wherein the therapeutically effective amount of a compound of the formula Ia or Ib is about 1 mg to about 100 mg per kg per day.

17. A pharmaceutical kit comprising one or more containers filled with a pharmaceutical composition according to claim 1, and a notice in the form prescribed by a governmental agency regulating the labeling, manufacture, use or sale of the pharmaceutical composition, which notice reflects approval by the agency of manufacture, use, or sale for human administration.

18. A pharmaceutical kit according to claim 17 wherein the notice reflects approval by the agency of manufacture, use, or sale for human administration in the treatment of Alzheimer's disease, dementia, or mild cognitive impairment.
